SEQUENCE LISTING (16 sequences)

SEQ ID NO: 1 - DNA, 20 bp, Artificial Sequence
  oligodeoxyribonucleotide
tccatgacgttcctgacgtt

SEQ ID NO: 2 - DNA, 20 bp, Artificial Sequence
  oligodeoxyribonucleotide
ataatcgacgttcaagcaag

SEQ ID NO: 3 - DNA, 20 bp, Artificial Sequence
  oligodeoxyribonucleotide
ataatagagcttcaagcaag

SEQ ID NO: 4 - DNA, 21 bp, Artificial Sequence
  oligodeoxyribonucleotide
tccaatgagcttcctgagtct

SEQ ID NO: 5 - DNA, 6 bp, Artificial Sequence
  oligodeoxyribonucleotide
gacgtt

SEQ ID NO: 6 - DNA, 20 bp, Artificial Sequence
  oligodeoxyribonucleotide
ggtgcatcgatgcagggggg

SEQ ID NO: 7 - DNA, 20 bp, Artificial Sequence
  oligodeoxyribonucleotide
ggtgcatcgatgcagggggg

SEQ ID NO: 8 - DNA, 27 bp, Artificial Sequence
  oligodeoxyribonucleotide
tcgtcgctgtctccgcttcttcttgcc

SEQ ID NO: 9 - DNA, 24 bp, Artificial Sequence
  oligodeoxyribonucleotide
tcgtcgttttgtcgttttgtcgtt

SEQ ID NO: 10 - DNA, 20 bp, Artificial Sequence
  oligodeoxyribonucleotide
atcgactctcgagcgttctc

SEQ ID NO: 11 - DNA, 12 bp, Artificial Sequence
  oligodeoxyribonucleotide
tcgagcgttctc

SEQ ID NO: 12 - DNA, 22 bp, Artificial Sequence
  oligodeoxyribonucleotide
tgactgtgaacgttcgagatga

SEQ ID NO: 13 - protein, 25 aa, Flavivirus
GGGSGGGGSGGGTGGGSGGGSGGGG

SEQ ID NO: 14 - protein, 55 aa, Flavivirus
GGGSGGGGSGGGTGGGSGGGSPRMKQLEDKVEELLSKNYHLENEVARLKKLVGER

SEQ ID NO: 15 - protein, 60 aa, Flavivirus
GGGSGGGGSGGGTGGGSGGGSPRMKQLEDKVEELLSKNYHLENEVARLKKLVGERGGCGG

SEQ ID NO: 16 - protein, 59 aa, Flavivirus
GGGSGGGGSGGGTGGGSGGGSPGELEELLKHLKELLKGPRKGELEELLKHLKELLKGEF

(19) United States
(12) Patent Application Publication
(10) Pub. No.: US 2004/0213808 A1
(43) Pub. Date: Oct. 28, 2004

(54) Recombinant vaccine against flavivirus infection

(76) Inventors: Michael Lieberman, Honolulu, HI (US); Tom Humphreys, Honolulu, HI (US)

Correspondence Address: MORRISON & FOERSTER LLP, 3811 VALLEY CENTRE DRIVE, SUITE 500, SAN DIEGO, CA, 92130-2332, US

(21) Appl. No.: 10/730,776

(22) Filed: Dec. 8, 2003

Related U.S. Application Data
(60) Provisional application No. 60/432,865, filed on Dec. 11, 2002.
(60) Provisional application No. 60/493,312, filed on Aug. 6, 2003.

(51) Int. Cl. A61K 39/193; A61K 31/704

(52) U.S. Cl. 424/218.1; 514/33; 514/44

(57) ABSTRACT

An immunogenic composition is described which preferably contains recombinantly produced forms of truncated flavivirus envelope glycoproteins and an adjuvant. The disclosed immunogenic compositions can further comprise a recombinantly produced non-structural (non-envelope) flavivirus protein. The adjuvant typically comprises a saponin preferably derived from the Quillaja saponica tree or a derivative thereof. The adjuvant can also comprise an oligodeoxyribonucleotide preferably containing specific sequences of nucleotides described herein. A pharmaceutically acceptable vehicle may also be included in the immunogenic composition.

### RELATED APPLICATION

This application claims the benefit of priority to U.S. Provisional Patent Application No. 60/432,865, filed Dec. 11, 2002, and to U.S. Provisional Patent Application No. 60/493,312, filed Aug. 6, 2003, both of which are hereby incorporated by reference in its entirety.

### TECHNICAL FIELD

The invention relates to an immunogenic formulation designed to elicit an immunological response against flaviviral infection. Specifically, the immunogenic formulation comprises at least one recombinant flavivirus envelope (E) glycoprotein produced in a cellular production system and an adjuvant. The immunogenic formulation may also comprise at least one recombinant flavivirus non-structural protein, preferably NS1. A preferred adjuvant comprises at least one saponin or derivative thereof, at least one oligodeoxyribonucleotide, or a combination of both. The disclosed immunogenic formulations induce higher titer virus neutralizing antibodies, and induce more potent cell-mediated immune responses, in comparison to conventional formulations.

### BACKGROUND

The family Flaviviridae includes the family prototype yellow fever virus (YF), the four serotypes of dengue virus (DEN-1, DEN-2, DEN-3, and DEN-4), Japanese encephalitis virus (JE), tick-bome encephalitis virus (TBE), West Nile virus (WN), Saint Louis encephalitis virus (SLE), and about 70 other disease causing viruses. Flaviviruses are small, enveloped viruses containing a single, positive-strand RNA genome. Ten gene products are encoded by a single open reading frame and are translated as a polyprotein organized in the order: capsid (C), “preMembrane” (prM, which is processed to “Membrane” (M) just prior to virion release from the cell), “envelope” (E), followed by non-structural (NS) proteins NS1, NS2a, NS2b, NS3, NS4a, NS4b and NS5 (reviewed in Chambers, T. J. et al., Annual Rev Microbiol (1990) 44:649-688; Henchal, E. A. and Putnak, J. R., Clin Microbiol Rev. (1990) 3:376-396). Individual flaviviral proteins are then produced through precise processing events mediated by host as well as virally encoded proteases.

The envelope of flaviviruses is derived from the host cell membrane, but contains the virally-encoded transmembrane envelope (E) glycoprotein. This E glycoprotein is the largest viral structural protein, and contains functional domains responsible for cell surface attachment and intraendosomal fusion activities. It is also a major target of the host immune system, inducing the production of virus neutralizing antibodies, which are associated with protective immunity.

Although the mode of flavivirus transmission and the pathogenesis of infection are quite varied among the different viruses, dengue viruses serve as an illustrative example of the family. Dengue viruses are transmitted to man by mosquitoes of the genus Aedes, primarily A. aegypti and A. albopictus. The viruses cause an illness manifested by high fever, headache, aching muscles and joints, and rash (Gibbons, R. V. and D. W. Vaughn, British Medical Journal (2002) 324:1563-1566). Some cases, typically in children, result in a more severe form of infection, dengue hemorrhagic fever/dengue shock syndrome (DHF/DSS), marked by severe hemorrhage, vascular permeability, or both, leading to shock. Without diagnosis and prompt medical intervention, the sudden onset and rapid progression of DHF/DSS can be fatal.

Dengue viruses are the most significant group of arthropod-transmitted viruses in terms of global morbidity and mortality with an estimated one hundred million cases of dengue fever occurring annually including 250,000 to 500,000 cases of DHF/DSS (Gubler, D. J., Clin. Microbiol. Rev. (1998) 11:480-496; Gibbons, supra). With the global increase in population, urbanization of the population especially throughout the tropics, and the lack of sustained mosquito control measures, the mosquito vectors of dengue have expanded their distribution throughout the tropics, subtropics, and some temperate areas, bringing the risk of dengue infection to over half the world's population. Modern jet travel and human emigration have facilitated global distribution of dengue serotypes, such that multiple serotypes of dengue are now endemic in many regions. Accompanying this there has been an increase in the frequency of dengue epidemics and the incidence of DHF/DSS in the last 15 years. For example, in Southeast Asia, DHF/DSS is a leading cause of hospitalization and death among children (Gubler, supra; Gibbons and Vaughn, supra).

While all dengue viruses are antigenically related, antigenic distinctions exist which define the four dengue serotypes. Infection of an individual with one serotype provides long-term immunity against reinfection with that serotype but fails to protect against infection with the other serotypes. In fact, immunity acquired by infection with one serotype may potentially enhance pathogenicity by other dengue serotypes. This is particularly troubling as secondary infections with heterologous serotypes have become increasingly prevalent as the virus has spread, resulting in the co-circulation of multiple serotypes in many geographical areas and increased numbers of cases of DHF/DSS (Gubler, supra). Regardless of the mechanism for enhanced pathogenicity of a secondary, heterologous dengue infection, strategies employing a tetravalent vaccine should avoid such complications. Helpful reviews of the nature of the dengue disease, the history of attempts to develop suitable vaccines, structural features of flaviviruses in general, as well as the structural features of the envelope protein of flaviviruses are available (Halstead 1988; Brandt, E. E., J. Infect Disease (1990) 162:577-583; Chambers, supra; Mandl, C. W. et al., Virology (1989) 63:564-571; Henchal and Putnak, supra; Gubler, supra; Cardosa, M. J., Brit. Med. Bull. (1998) 54:395-405).

While a significant amount of effort has been invested in developing candidate live-attenuated dengue vaccine strains, many strains tested have proven unsatisfactory (see, e.g., Eckels, K. H. et al., Am. J. Trop. Med. Hyg. (1984) 33:684-689; Bancroft, W. H. et al., Vaccine (1984) 149:1005-1010; McKee, K. T., et al., Am. J. Trop. Med. Hyg. (1987) 36:435-442). Despite this limited success, live attenuated candidate vaccine strains continue to be developed and tested (Bhamarapravati, N. et al., Bull. World Health Organ. (1987) 65:189-195; Hoke, C. H., Jr. et al., Am. J. Trop. Med. Hyg. (1990) 43:219-226; Angsubhakorn, S., et al., Southeast Asian J. Trop. Med. Public Health (1994) 25:554-559; Dharakul, T. et al., J. Infect. Dis. (1994) 170:27-33; Edelman, R. et al., J. Infect. Dis. (1994) 170:1448-1455; Vaughn, D. W. et al., Vaccine (1996) 14:329-336; Bhamarapravati, N., and Sutee, Y., Vaccine (2000) Suppl 2:44-47; Kanesa-thasan, N. et al., Vaccine (2001) 19:3179-3188; Sabchareon, A. et al., Am. J. Trop. Med. Hyg. (2002) 66:264-272). Another approach to development of a live vaccine for dengue is a recombinant chimeric (intertypic) dengue vaccine (Bray, M. et al., J. Virol. (1996) 70:4162-4166; Chen, W., et al., J. Virol. (1995) 69:5186-5190; Bray, M. and Lai, C.-J., Proc. Natl. Acad. Sci. USA (1991) 88:10342-10346; Lai, C. J. et al., Clin. Diagn. Virol. (1998) 10:173-179). However, all of the live virus vaccine approaches remain plagued by difficulties in developing properly attenuated strains and in achieving balanced, tetravalent formulations.

Similarly, efforts to develop killed dengue vaccines have met with limited success. Primarily these studies have been limited by the inability to obtain adequate viral yields from cell culture systems. Virus yields from insect cells such as C6/36 cells are generally in the range of 10⁴ to 10⁵ pfu/ml, well below the levels necessary to generate a cost-effective killed vaccine. Yields from mammalian cells including LLC-MK2 and Vero cells are higher, but the peak yields, approximately 10⁸ pfu/ml from a unique Vero cell line, are still lower than necessary to achieve a truly cost-effective vaccine product.

In the absence of effective live attenuated or killed dengue vaccines, a significant effort has been invested in the development of recombinant dengue subunit vaccines. Many of the vaccine efforts that use a recombinant DNA approach have focused on the E glycoprotein. This glycoprotein is a logical choice for a subunit vaccine as it plays a central role in the biology and the host immune response to the virus. The E glycoprotein is exposed on the surface of the virus, binds to the cell receptor, and mediates fusion (Chambers, supra). It has also been shown to be the primary target for the neutralizing antibody response (Mason, P. W., J. Gen Virol (1989) 70:2037-2048). Monoclonal antibodies directed against purified flaviviral E proteins are neutralizing in vitro and some have been shown to confer passive protection in vivo (Henchal, E. A. et al., Am. J. Trop. Med. Hyg. (1985) 34:162-169; Heinz, F. X. et al., Virology (1983) 130:485-501; Kimura-Kiroda, J. and Yasui, K., J. Immunol. (1988) 141:3606-3610; Trirawatanapong, T. et al., Gene (1992) 116:139-150).

While many heterologous expression systems have been developed and shown to be effective for production of certain recombinant products, not all expression systems are effective for producing all recombinant products. In fact, despite the fact that a system may be reported to be effective for production of one recombinant protein, predictions on efficacy of expression of other recombinant products do not always hold. In particular, efficient expression of conformationally relevant recombinant flavivirus E has remained elusive. A wide variety of expression systems ranging from bacterial, fungal, and insect to mammalian systems have failed to efficiently produce conformationally relevant flavivirus E in significant quantities, highlighting the highly empirical nature of efficient heterologous gene expression.

Much progress in the analysis and understanding of the immune response to foreign antigens has been made in the last decade or two, particularly in the realm of cellular immunology. The delineation of subsets of lymphocytes with distinct functional properties and the characterization of the interactions between these subsets of cells has provided detailed mechanistic explanations for the overall functioning of the immune system. One central paradigm that has emerged revolves around the description of two classes of T “helper” lymphocytes, termed “Th1” and “Th2” cells (Table 1). These two classes of T cells are primarily distinguished by the pattern of cytokine expression elaborated by each. The cytokines produced by Th1 cells (IFN-γ, IL-2, TNF-β) tend to promote the cellular immune effector response required to combat parasitic, fungal, and intracellular viral agents (Moingeon, P., J. Biotechnol. (2002) 98:189-198). The cytokines produced by Th2 cells (IL-4, IL-5, IL-6, IL-10, IL-13), tend to promote antibody synthesis, i.e., the humoral immune effector response. These antibodies are effective in controlling extracellular bacterial pathogens. The balance between Th1 and Th2 cytokines is a dynamic one, because of the fact that Th1 cytokines tend to inhibit the production of Th2 cytokines in vivo, and vice versa. Thus, a viral vaccine capable of stimulating a “Th1” type immune response (in addition to stimulation of antibody production) would reasonably be expected to be more efficacious in protection against infection than a vaccine eliciting only an antibody response.

TABLE 1
T helper type 1 (Th1) and T helper type 2 (Th2) lymphocytes

| | TH1 | |
|---|---|---|
| CHARACTERISTIC | LYMPHOCYTES | TH2 LYMPHOCYTES |
| Cytokines produced | IFN-γ, TNF-β, | IL-4, IL-5, IL-6, IL-10, |
| | IL-2 | IL-13 (IL-4 is particularly |
| | | important for IgE synthesis) |
| Type of associated | CELL-MEDIATED | Humoral |
| immune response | | |
| Associated antibody | IgG_{2A} (MOUSE) | IgG₁, IgE |
| isotypes | | |

Adjuvants are materials that increase the immune response to a given antigen. Since the first report of such an enhanced immunogenic effect by materials added to an antigen (Ramon, G., Bull. Soc. Centr. Med. Vet. (1925) 101:227-234), a large number of adjuvants have been developed, but only calcium and aluminum salts are currently licensed for use in human vaccine products. Numerous studies have demonstrated that other adjuvants are significantly more efficacious for inducing both humoral and cellular immune responses. However, most of these have significant toxicities or side-effects which make them unacceptable for human and veterinary vaccines. In fact, even aluminum hydroxide has recently been associated with the development of injection site granulomas in animals, raising safety concerns about its use. Because of these problems significant efforts have been invested in developing highly potent, but relatively non-toxic adjuvants. A number of such adjuvant formulations have been developed and show significant promise (Cox, J. C. and Coulter, A. R., Vaccine (1997) 15:248-256; Gupta, R. K. and Siber, G. R., Vaccine (1995) 13:1263-1276), especially in combination with recombinant products. Several of these modern adjuvants are being tested in preclinical and clinical trials designed to examine both efficacy and safety. The modes of action of adjuvants include (i) a depot effect, (ii) immunomodulation, (iii) targeting specific antigen-presenting cell populations, (iv) formation of micelles or liposomes, and (v) maintaining appropriate “native” conformation of the antigen. The depot effect results from either the adsorption of protein antigens onto aluminum gels or the emulsification of aqueous antigens in water-in-oil emulsions. In either case this results in the subsequent slow release of these antigens into the circulation from local sites of deposition. This prevents the rapid loss of most of the antigen that would occur by passage of the circulating antigen through the liver. Immunomodulation involves stimulation of the “innate” immune system through interaction of particular adjuvants with cells such as monocytes/macrophages or natural killer (NK) cells. These cells become activated and elaborate proinflammatory cytokines such as TNF-α and IFN-γ, which in turn stimulate T lymphocytes and activate the “adaptive” immune system. Bacterial cell products, such as lipopolysaccharides, cell wall derived material, DNA, or oligonucleotides often function in this manner (Krieg, A. M. et al., Nature (1995) 374:546; Ballas, Z, J, et al., J. of Immunology (2001) 167:4878-4886; Chu, R. S., et al., J. Exp. Med. (1997) 186:1623; Hartmann, G. and Krieg, A., J. Immunol. (2000) 164:944-952; Hartmann, G., et al., J. of Immunol. (2000) 164:1617-1624; Weeratna, R. D. et al., Vaccine (2000) 18:1755-1762; U.S. Pat. Nos. 5,663,153; 5,723,335; 6,194,388; 6,207,646; 6,214,806; 6,218,371; 6,239,116; 6,339,068; 6,406,705; 6,429,199). Modes of action iii, targeting specific antigen-presenting cell populations, and iv, formation of micelles or liposomes, are discussed below in regards to adjuvants.

Finally, some adjuvants may have the ability to maintain the antigen in its “native” conformation, thereby protecting important “conformational” epitopes. These epitopes may be important for eliciting the production of antibodies with particular functional capabilities, such as viral neutralization.

It would be useful to discover a flavivirus vaccine or immune composition used in combination with an adjuvant that induces higher titer neutralizing antibodies and more potent cell-mediated immune responses in comparison to conventional combinations.

### SUMMARY OF THE INVENTION

The disclosed invention provides immunogenic compositions containing as active ingredients recombinantly-produced forms of truncated flavivirus envelope and non-structural glycoproteins. A preferred embodiment of the disclosed invention also includes an adjuvant, such as saponin or a saponin-like material, either alone or in combination with an oligodeoxyribonucleotide (ODN), as a component of the immunogenic formulations described herein. Typically, the disclosed immunogenic formulations are capable of eliciting the production of neutralizing antibodies against dengue viruses and stimulating cell-mediated immune responses.

Other aspects of this invention include: use of a therapeutically effective amount of the immunogenic composition in an acceptable carrier for use as an immunoprophylactic against flavivirus infection and a therapeutically effective amount of the immunogenic composition in an acceptable carrier as a pharmaceutical composition.

### BRIEF DESCRIPTION OF THE DRAWINGS

FIG. 1A. Coomassie blue stained SDS-PAGE of West Nile 80E protein expressed by Drosophila S2 cells under non-reducing conditions. Lane 1) Spinner Culture #1 of cell line WN-80E-1 harvested Feb. 19, 2003, Lane 2) Spinner Culture #2 of cell line WN-80E-1 harvested Feb. 10, 2003, Lane 3) Culture of a dengue transformant cell line. The migration of the West Nile 80E is faster than the dengue 80E due to differences in glycosylation and tertiary structure (samples are non-reduced).

FIG. 1B. Western blot of duplicate SDS-PAGE gel seen in FIG. 1A. The blot was probed with a commericially available West Nile rabbit polyclonal from BioReliance. This antibody cross-reacts slightly with the Dengue 80E.

FIG. 2A. Coomassie blue stained SDS-PAGE of West Nile NS1 protein expressed by Drosophila S2 cells under reducing (Lanes 1 and 2) and non-reducing conditions (Lanes 3 and 4). Lanes 1 and 3) Spinner Culture #1 of cell line WN-NS1-5 harvested Jul. 6, 2003, Lanes 2 and 4) Spinner Culture #2 of cell line WN-NS1-5 harvested Jul. 6, 2003.

FIG. 2B. Western blot of duplicate SDS-PAGE gel seen in FIG. 2A. The blot was probed with the mouse monoclonal 7E11. The two approximately 40 kD bands of WN-NS1 are two different glycoforms of the NS1 protein. The higher MW reactive band at about 80 kD in lanes 3 and 4 is a dimer. The 7E11 antibody reacts more strongly with reduced than non-reduced NS1.

FIG. 3. Coomassie stained SDS-PAGE gel (A) and Western blot (B) of purified West Nile 80E. Both samples were run under non-reducing conditions on 10% gels. The Western blot was developed using a rabbit polyclonal antisera developed against formalin inactivated dengue virus. The sizes of the molecular weight markers (in kD) are indicated to the left of the gel and blot. The sample loadings (in μg) are presented at the top of each.

FIG. 4 Coomassie stained SDS-PAGE gel (A) and Western blot (B) of purified West Nile NS1. Both samples were run under non-reducing conditions on 10% gels. The Western blot was developed using a rabbit polyclonal antisera developed against purified dengue NS1. The sizes of the molecular weight markers (in kD) are indicated to the left of the gel and blot. The sample loadings (in μg) are presented at the top of each.

FIG. 5 is a bar graph demonstrating the effect of the addition of NS1 to an 80% E dengue vaccine on the production of interferon-γ (IFN-γ) in vitro by splenocytes from mice immunized with the vaccine of the invention and stimulated in vitro with the vaccine antigens.

FIG. 6. Antigen-stimulated lymphocyte proliferation. Experimental procedure as described in Example 8 below. Results shown represent the mean of 2 mice from each vaccinee group. Net cpm calculated as the mean cpm of quadruplicate antigen-stimulated wells minus the mean cpm of quadruplicate unstimulated wells (cell controls without antigen). Unstimulated cpm=2792 and 2012 for the groups vaccinated with antigens plus QS-21 and QS-21+CpG, respectively. Data from mice vaccinated and stimulated with 80E prepared in PBS with tween are shown. Data from mice vaccinated and/or stimulated with 80E prepared in PBS without tween were similar.

FIG. 7. Antigen-stimulated IFN-γ production. Experimental procedure as described in Example 8 below. Results depict the values obtained from one mouse, representative of each vaccinee group. Unstimulated cell controls yielded undetectable levels of IFN-γ (<0.05 ng/ml). Data from mice vaccinated and stimulated with 80E prepared in PBS with tween are shown. Data from mice vaccinated and/or stimulated with 80E prepared in PBS without tween were similar.

FIG. 8. Antigen-stimulated IL-10 production. Experimental procedure as described in Example 8 below. Results depict the values obtained from one mouse, representative of each vaccinee group. Unstimulated cell controls yielded undetectable levels of IL-10 (<0.05 ng/ml). Data from mice vaccinated and stimulated with 80E prepared in PBS with Tween are shown. Data from mice vaccinated and/or stimulated with 80E prepared in PBS without Tween were similar.

### DESCRIPTION OF THE PREFERRED EMBODIMENTS

The invention described herein provides a subunit flavivirus immunogenic formulation that is produced and secreted using a recombinant expression system, preferably combined with one or more adjuvants. The disclosed immunogenic formulations are effective in inducing a strong virus neutralizing antibody response to Flaviviruses as well as stimulating cell-mediated immune responses to the viruses.

In accordance with the invention, the disclosed immunogenic compositions may include an adjuvant. A preferred adjuvant is a saponin or a saponin-derivative or saponin-like substance, preferably QS-21 U.S. Pat. Nos. 5,057,540; 5,583,112; 6,231,859) with an oligodeoxyribonucleotide (ODN) (U.S. Pat. Nos. 5,663,153; 5,723,335; 6,194,388; 6,207,646; 6,214,806; 6,218,371; 6,239,116; 6,339,068; 6,406,705; 6,429,199).

The antigens used in the disclosed immunogenic compositions typically comprise a flavivirus envelope protein and a non-structural protein. For example, a preferred immunogenic composition comprises a Drosophila cell-expressed envelope protein (preferably 80% E). Preferably envelope protein subunits from each of the four dengue virus serotypes are used in the composition (see U.S. Pat. Nos. 6,136,561; 6,165,477; 6,416,763; 6,432,411; U.S. patent application Ser. No. 08/904,227, filed Jul. 31, 1997). Envelope proteins from other flaviviruses such as Japanese encephalitis virus (JE), tick-borne encephalitis virus (TBE), West Nile virus (WN), and Saint Louis encephalitis virus (SLE) are also contemplated for use with the disclosed invention.

Additionally, a recombinant flavivirus non-structural protein is included in the disclosed immunogenic composition. For example, a Drosophila cell-expressed non-structural protein (preferably NS1), preferably from dengue serotype 2 (U.S. Pat. No. 6,416,763) is included in the disclosed immunogenic compositions. Inclusion of these components typically results in an exceptionally potent vaccine formulation.

The combination of viral structural and non-structural proteins and one or more adjuvants induces very high titer neutralizing antibodies in mice. For example, the use of a saponin-like material alone, preferably QS-21, as adjuvant with the same recombinant antigens yields a high, but slightly lower titer of virus neutralizing antibodies. The cell-mediated response (correlated with the production of IFN-γ from immune splenocytes by antigenic stimulation in vitro) is significantly enhanced when QS-21 is used with an oligodeoxyribonucleotide as the adjuvants. In contrast, the combination of the same recombinant antigens with other modern adjuvants (e.g., aluminum salts or MF59) failed to induce such a potent immune response suggesting the uniqueness of the combination. Examples illustrating the efficacy of the unique combination are contained herein below. 1) Envelope Protein Subunits

(a) 80% E

In the most preferred embodiment of the invention, the recombinant protein components of the flavivirus vaccine formulations described herein are produced by an alternative eukaryotic expression system, Drosophila melanogaster Schneider 2 (S2) cells (Johansen, H. et al., Genes Dev. (1989) 3:882-889; Ivey-Hoyle, M., Curr. Opin. Biotechnol. (1991) 2:704-707; Culp, J. S., et al., Biotechnology (NY) (1991) 9:173-177). This method of expression has shown to be successful to produce recombinant envelope proteins from Flaviviruses, such as dengue serotypes 1-4 and Japanese encephalitis virus (JE). These proteins are truncated at the C-terminus, leaving approximately 80% of the native envelope protein (80% E). The truncation deletes the membrane anchor portion of the protein, thus allowing it to be secreted into the extracellular medium, facilitating recovery. Furthermore, the expressed proteins have been shown to be properly glycosylated and to maintain native conformation as determined by reactivity with a panel of conformationally sensitive monoclonal antibodies, 4G2 and 9D12, (Coller, B G, Clements, DE, Bignami, GS, et al., Hawaii Biotech, unpublished data; U.S. Pat. Nos. 6,136,561 and 6,165,477).

As such, in another embodiment of the invention, 80% E is defined more broadly as an envelope protein subunit that comprises six disulfide bridges at Cys1-Cys2, Cys3-Cys8, Cys4-Cys6, Cys5-Cys7, Cys9-Cys10 and Cys11-Cys12; wherein the polypeptide has been secreted as a recombinant protein from Drosophila cells; and wherein the polypeptide generates neutralizing antibody responses to a homologous strain of a species of Flavivirus in a murine host.

In a more preferred embodiment, the envelope protein subunit further comprises a hydrophilicity profile characteristic of a homologous 80% portion of an envelope protein (80% E) starting from the first amino acid at the N-terminus of the envelope protein of a strain of a species of Flavivirus.

In an even more preferred embodiment, the hydrophilicity profile characteristic of 80% E confers the same secondary and tertiary structures as the homologous 80% E.

The immunogenicity and protective efficacy of such truncated E proteins have also been amply demonstrated in animal models (U.S. Pat. Nos. 6,136,561; 6,165,477; 6,416,763; 6,432,411; Jan, L., et al., Am. J. Trop. Med. Hyg., 48(3), (1993) pp. 412-423; Men, R. et al., J. Virol (1991) 65:1400-1407).

As previously described (Ivy et al., U.S. Pat. No. 6,136,561; Ivy et al., U.S. Pat. No. 6,165,477; McDonnell et al., U.S. Pat. No. 6,416,763; Ivy et al., U.S. Pat. No. 6,432,411) and, used herein, “80% E” in one instance refers to a polypeptide that spans a flavivirus envelope protein, preferably of one of approximately the first 395 amino acids starting from the N-terminal amino acid of the envelope protein, such as from amino acids 1-395 thereof.

Preferably, the envelope protein subunit is a portion of the dengue envelope protein (E) that comprise approximately 80% of its length starting from amino acid residue 1 at its N-terminus and which portion has been recombinantly produced and secreted from Drosophila cells. In another embodiment, 80% E is at least 80%, or 85%, or 90% or 95% homologous over the entire sequence relative to native flavivirus 80% E. More preferably, 80% E is derived from each of the four broader serotypes of the dengue virus, and to homologs or variants as described above. For example, serotypes of dengue virus such as: DEN-1; DEN-2; DEN-3; and DEN-4, as well as any serotypes of: Japanese encephalitis virus (JE), Tick-borne encephalitis virus (TBE), West Nile virus (WN), Saint Louis encephalitis virus (SLE), and the family prototype, Yellow fever virus (YF) are included. The 80% E proteins preferably are produced from vectors containing the DNA encoding the dengue virus prM as a fusion with 80% E. The fusion protein is processed by cellular enzymes to release the mature 80% E proteins.

In one embodiment, the immunogenic composition comprises one or more of the four envelope protein subunits derived from dengue virus serotypes 1, 2, 3, and 4. Preferably the immunogenic composition comprises all four envelope protein subunits derived from the dengue serotypes. Preferably, the 80% E subunits from each serotype of dengue virus are purified by immunoaffinity chromatography (IAC) using a monoclonal antibody (4G2) as previously described (Ivy et al., U.S. Pat. No. 6,432,411, example 9).

(b) Dimeric 80% E

Numerous studies have demonstrated that immunogenicity is directly related both to the size of the immunogen and to the antigenic complexity of the immunogen. Thus, in general, larger antigens make better immunogens. The native form of E protein found on the surface of the flavivirus virion is a homodimer (Rey F. A. et al., Nature (1995) 375:291-298). The recombinant dengue E protein discussed above is monomeric and therefore is not identical to the natural viral E protein. Thus, in an attempt to produce a protective recombinant flavivirus immunogenic formulation, preferably an immunogenic formulation protective against dengue virus infection, with enhanced immunogenicity, dimerized versions of the E proteins were produced by genetic engineering techniques (Coller, B G, Clements, D E, Bignami, G S, et al., Hawaii Biotech, unpublished data; U.S. patent application Ser. No. 08/904,227, filed Jul. 31, 1997). In a preferred embodiment, at least one envelope protein subunit from dengue is a dimer.

The modifications that can be made to the 80% E products by addition of carboxy-terminal sequences encoding flexible linkers, leucine zipper domains, or four helix bundle domains, designed to enhance the dimerization of the 80% E molecules, are described in detail below. All of these dimeric 80% E proteins are produced from vectors containing the DNA encoding the flavivirus prM as a fusion with mature proteins resulting in secretion of the processed, mature dimeric 80% E proteins from which the prM protein has been removed.

Three basic approaches have been disclosed in U.S. patent application Ser. No. 09/376,463 to construct dimeric 80% E molecules. The first approach involves using tandem copies of 80% E covalently attached to each other by a flexible linker. In a preferred embodiment, “Linked 80% E Dimer” refers to a polypeptide which encodes DEN-2 80% E-GGGSGGGGSGGGTGGGSGGGSGGGG-DEN-2 80% E (SEQ ID NO: 13). The stretch of amino acids covalently linking the two copies of DEN2 80% E is designed to serve as a flexible tether allowing the two 80% E molecules to associate in native head-to-tail dimeric orientation while maintaining their covalent attachment to each other. “Linked 80% E Dimer” also refers to the corresponding peptide region of the envelope protein of the others three dengue serotypes, homologs and to any naturally occurring variants, as well as corresponding peptide regions of the envelope (E) protein of other Flaviviruses. For example, serotypes of dengue virus such as: DEN-1; DEN-2; DEN-3; and DEN-4, as well as any serotypes of: JE, TBE, WN, SLE and YF are included.

It would be readily apparent to one of ordinary skill in the art to select other linker sequences as well. The portion of present invention directed to dimeric molecules is not limited to the specific disclosed linkers, but, to any amino acid sequence that would enable the two 80% E molecules to associate in native head to tail dimeric orientation while maintaining their covalent attachment to each other.

The second approach involves addition of a carboxy-terminal leucine zipper domain to monomeric 80% E to enhance dimerization between two 80% E-leucine zipper molecules. Two versions of this approach have been adopted. One version includes a disulfide bond linking the leucine zipper domains resulting in a covalently linked dimer product, while the other is based on the non-covalent association of the leucine zipper domains. As used herein “80% E ZipperI” refers to a polypeptide that, in association with another polypeptide, produces a non-covalently linked dimer, and preferably refers to a polypeptide which encodes DEN-2 80% E-GGGSGGGGSGGGTGGGSGGGSPRMKQLEDKVEELLSKNYHLENEVARLKKLVGER (SEQ ID NO: 14). The first 22 amino acids extending after the carboxy terminus of 80% E serve as flexible tether between 80% E and the adjacent leucine zipper domain. The leucine zipper domain is designed to dimerize with the identical sequence from another 80% E Zipper molecule. The formation of a non-covalently linked leucine zipper will enhance the dimerization of the 80% E molecules, which may associate in native head to tail conformation by virtue of the flexible linker connecting the 80% E molecules with the leucine zipper domain. “80% E ZipperI” also refers to the corresponding peptide region of the envelope protein of the three other dengue serotypes, and to homologs or any naturally occurring variants, as well as corresponding peptide regions of the envelope (E) protein of other flaviviruses. For example, serotypes of dengue virus such as: DEN-1; DEN-2; DEN-3; and DEN-4, as well as any serotypes of: JE, TBE, WN, SLE and YF.

It would be readily apparent to one of ordinary skill in the art to select other leucine zipper sequences as well. The present invention is not limited to the specific disclosed leucine zipper sequences, but to any amino acid sequences that would enable the dimerization between identical sequences from another 80% E Zipper molecule.

As used herein “80% E ZipperII” refers in one instance to a polypeptide that, in association with another polypeptide, produces a covalently linked dimer and preferably to a polypeptide which encodes DEN-2 80% E-GGGSGGGGSGGGTGGGSGGGSPRMKQLEDKVEELLSKNYHLENEVARLKKLVGERGGCGG (SEQ ID NO: 15). The first 22 amino acids extending after the carboxy terminus of 80% E serve as flexible tether between 80% E and the adjacent leucine zipper domain. In one preferred embodiment, the method of making a “ZipperII” dimer involves addition of a carboxy-terminal peptide linker (“flexible tether”) to a “leucine zipper” peptide sequence which forms a helical secondary structure. The leucine zipper helical structure dimerizes (non-covalently associates) with another identical leucine zipper sequence on another E protein subunit molecule. The leucine zipper domain of 80% E ZipperII is further modified (engineered) to contain a glycine-glycine-cysteine-glycine-glycine peptide sequence at its carboxy terminus (GGCGG sequence) which facilitates disulfide bond formation between the cysteine residues within the two leucine zipper helices. Thus, once the leucine zipper dimerizes, a disulfide bond forms between the two ends, resulting in a covalently linked dimer product. The formation of a covalently linked leucine zipper results in the dimerization of the 80% E molecules, which may associate in native head to tail conformation by virtue of the flexible linker connecting the 80% E molecules with the leucine zipper domain. “80% E ZipperII” also refers to the corresponding peptide region of the envelope protein of the three other dengue serotypes, and to any naturally occurring variants, as well as corresponding peptide regions of the envelope (E) protein of other Flaviviruses. For example, serotypes of dengue virus such as: DEN-1; DEN-2; DEN-3; and DEN-4, as well as any serotypes of: JE, TBE, WN, SLE and YF. DEN-4 80% E Zipper II containing a GGCGG sequence is especially preferred.

It would be readily apparent to one of ordinary skill in the art to select other leucine zipper sequences as well. The present invention is not limited to the specific disclosed leucine sequences, but to any amino acid sequences that would permit the dimerization with an identical sequence from another 80% E Zipper molecule. Further, the ordinary skilled artisan would readily be able to determine other sequences that would facilitate disulfide bond formation between the two leucine zipper helices.

Another approach used to enhance dimerization of 80% E is the addition of a helix-turn-helix domain to the carboxy terminal end of 80% E. The helix-turn-helix domain from one modified 80% E molecule will associate with that of another to form a dimeric four-helix bundle domain. Preferably, an “80% E Bundle” refers to such a dimeric four-helix bundle domain and preferably to a polypeptide which encodes DEN-2 80% E-GGGSGGGGSGGGTGGGSGGGSPGELEELLKHLKELLKGPRKGELEELLKHLKELLKGEF (SEQ ID NO: 16). The first 22 amino acids extending after the carboxy terminus of 80% E serve as flexible tether between the 80% E domain and the helix-turn-helix domain which follows. The formation of a non-covalently associated four-helix bundle domain will enhance the dimerization of the 80% E molecules which may associate in the native head to tail conformation by virtue of the flexible linkers connecting 80% E to the helix bundle. “80% E Bundle” also refers to the corresponding peptide region of the envelope protein of the three remaining dengue serotypes, and to any naturally occurring variants, as well as corresponding peptide regions of the envelope (E) protein of other Flaviviruses. For example, serotypes of dengue virus such as: DEN-1; DEN-2; DEN-3; and DEN4, as well as any serotypes of: JE, TBE, WN, SLE and YF.

It would be readily apparent to one of ordinary skill of the art to select other amino acid sequences that would form the flexible tether extending after the carboxy terminal of the 80% E and also comprising a helix-turn-helix domain. The present invention is not limited to the specific disclosed helix-turn-helix domains, but to any amino acid sequences that would enable the dimerization of one modified 80% E molecule through a non-covalent association with a second modified 80% E molecule. Further, the ordinary skilled artisan would readily be able to determine other sequences that would facilitate such non-covalent association of helices.

2) Flavivirus Non-Structural Subunits

In addition to the flavivirus envelope proteins discussed above, the immunogenic formulations of the described invention preferably include a flavivirus non-structural protein. Flavivirus non-structural (NS) proteins may include: NS1, NS2a, NS2b, NS3, NS4a, NS4b and NS5 (Chambers, supra; Henchal and Putnak, supra). In a preferred embodiment, the non-structural protein is NS1 from dengue virus and is recombinantly expressed and secreted from Drosophila host cells, preferably Drosophila melanogaster Schneider (S2) cells as described in U.S. Pat. No. 6,416,763. Including a non-structural protein such as NS1 in the vaccine enhances the ability of the vaccine to elicit a cell-mediated immune response in the vaccinee, as well as an additional humoral component of immunity. Although non-structural proteins are not present in mature virions, they are produced in infected cells as a necessary part of the enzymatic system for viral replication (Mackenzie, J. M. et al., Virol., (1996) 220:232-240). Peptide epitopes processed from these proteins are displayed on the surface of infected antigen-presenting cells in association with MHC class I molecules, and thus may be recognized by a subset of immune cell populations, i.e., CD8+ T lymphocytes. When activated, these cells can function as cytotoxic T cells, and thus are capable of eliminating cells infected with virus (Cane, P. A. et al., J. Gen. Virol., (1988) 69:1241-1246; Livingston, P. G., et al., J. Immunol. (1995) 154:1287-1295; Mathew, A. et al., J. Clin. Invest. (1996) 98:1684-1692). This cellular immune response contributes to the overall protective efficacy of a subunit vaccine. Indeed, the protective efficacy of immunization with NS1 has been demonstrated for several Flaviviruses (Falgout, B. et al., J. Virol., (1990) 64(9):4356-4363; Fleeton, M. N. et al., J. Gen. Virol (1999) 80:1189-1198; Hall, R. A. et al., J. Gen. Virol., (1996) 77:1287-1294; Henschal, Henschal, and Schlesinger, 1988; Jacobs, S. C., et al., J. of Gen. Virol. (1994) 75:2399-2402). In addition, there is evidence that NS1 may elicit a humoral protective immune response involving the complement fixing activity of antibodies to this protein through mechanisms such as antibody-dependent, complement-mediated cytolysis, or Fc receptor mediated antibody-dependent cellular cytotoxicity (ADCC) (Putnak and Schlesinger, 1990; Schlesinger, J. J. et al., J. Immunol., (1985) 135(4):2805-2809; Schlesinger, J. J. et al., J. Virol., (1986) 60(3):1153-1155; Schlesinger, J. J., et al., J. Gen. Virol. (1987) 68:853-857; Schlesinger, J. J. et al., J. Gen. Virol. (1990) 71:593-599; Schlesinger, J. J. et al., Virology (1993) 192:132-14). Thus, the inclusion of a flavivirus non-structural protein such as NS1 in the candidate vaccine can be justified on the basis of a humoral as well as a cellular immune response.

In a preferred embodiment, the NS1 subunit from dengue serotype 2 (but may be from any of the four serotypes of dengue virus or any other flavivirus) produced by the Drosophila S2 cell expression system described above is also purified by IAC, but using a different monoclonal antibody (7E11), as previously described (McDonnell et al., U.S. Pat. No. 6,416,763, example 6).

3) Adjuvant

(a) Saponin

Targeting specific antigen-presenting cell (APC) populations, listed above as one of the modes of action of adjuvants, may involve a particular receptor on the surface of the APC, which could bind the adjuvant/antigen complex and thereby result in more efficient uptake and antigen processing as discussed above. For example, a carbohydrate-specific receptor on an APC may bind the sugar moieties of a saponin such as QS-21 (Kensil, C. R. et al., J. Immunol. (1991) 146:431-437; Newman M. J. et al., J. Immunol. (1992) 148:2357-2362; U.S. Pat. Nos. 5,057540; 5,583,112; 6,231,859). Although the validity of the invention is not bound by this theory, a possible mechanism of action may be that if the saponin is also bound to an antigen, this antigen would thus be brought into close proximity of the APC and more readily taken up and processed. Similarly, if the adjuvant forms micellar or liposomal complexes with antigen and the adjuvant can interact or fuse with the APC membrane, this may allow the antigen access to the cytosolic or endogenous pathway of antigen processing. As a result, peptide epitopes of the antigen may be presented in the context of MHC class I molecules on the APC, thereby inducing the generation of CD8+ cytotoxic T lymphocytes (CTL; Newman et al., supra; Oxenius, A., et al., J. Virol. (1999) 73: 4120).

A saponin is any plant glycoside with soapy action that can be digested to yield a sugar and a sapogenin aglycone. Sapogenin is the nonsugar portion of a saponin. It is usually obtained by hydrolysis, and it has either a complex terpenoid or a steroid structure that forms a practicable starting point in the synthesis of steroid hormones. The saponins of the invention can be any saponin as described above or saponin-like derivative with hydrophobic regions, especially the strongly polar saponins, primarily the polar triterpensaponins such as the polar acidic bisdesmosides, e.g. saponin extract from Quillsjabark Araloside A, Chikosetsusaponin IV, Calendula-Glycoside C, chikosetsusaponin V, Achyranthes-Saponin B. Calendula-Glycoside A, Araloside B, Araloside C, Putranjia-Saponin III, Bersamasaponiside, Putrajia-Saponin IV, Trichoside A, Trichoside B, Saponaside A, Trichoside C, Gypsoside. Nutanoside, Dianthoside C, Saponaside D, aescine from Aesculus hippocastanum or sapoalbin from Gyposophilla struthium, preferably, saponin extract Quillaja saponaria Molina and Quil A. In addition saponin may include glycosylated triterpenoid saponins derived from Quillaja Saponaria Molina of Beta Amytin type with 8-11 carbohydrate moieties as described in U.S. Pat. No. 5,679,354. Saponins as defined herein include saponins that may be combined with other materials, such as in an ISCOM-like structure as described in U.S. Pat. No. 5,679,354. Saponins also include saponin-like molecules derived from any of the above structures, such as GPI-0100, such as described in U.S. Pat. No. 6,262,029.

Preferably, the saponins of the invention are amphiphilic natural products derived from the bark of the tree, Quillaia saponaria. Preferably, they consist of mixtures of triterpene glycosides with an average molecular weight (M_{w}) of 2000. The most preferred embodiment of the invention is a purified fraction of this mixture (QS-21), which is a water-soluble, quillaic acid-based triterpene, with an acylated 3,28-O-bisglycoside structure, with good water solubility, and the ability to form micelles at neutral pH.

(b) Oligodeoxyribonucleotide

Synthetic oligodeoxynucleotides (ODNs) containing unmethylated cytosine-guanosine dinucleotides (CpG-ODNs) stimulate immune system cells. Optimally active K-type ODNs have a phosphorothioate backbone and express multiple unmethylated CpG dinucleotides flanked by a 5′ thymidine (T) and a TpT or ApT dinucleotide at the 3′-flanking position. D-type ODNs are structurally complex. Optimally active D-type ODNs contain a cental purine/pyrimidine/CpG/purine/pyrimidine motif flanked on both sides by 3-4 self-complementary bases. (See Verthelyi & Klinman, Clinical Immunology, 109:64-71 (2003).)

In vitro, CpG-ODNs directly activate B cells and plasmacytoid dendritic cells. CpG-ODNs have also been reported to indirectly activate monocytes, macrophages, NK cells, and memory T cells. In vivo, CpG-ODNs have been reported to be potent adjuvants that promote cellular and humoral immune responses. For example, particularly encouraging results have been reported in a study of an oligonucleotide adjuvant with a recombinant subunit viral vaccine (hepatitis B vaccine) in humans. The reported combination showed that the adjuvant enhanced the immune response to the vaccine, while being well-tolerated, both locally and systemically. Those of ordinary skill in the art will recognize, however, that the efficacy of any given adjuvant is immunogen dependent and thus predicting which combinations will be successful is difficult.

In a preferred embodiment, an immunostimulatory oligonucleotide is synthetic, between 2 to 100 base pairs in size and contains a consensus mitogenic CpG motif represented by the formula:

5′X₁X₂CGX₃X₄3′

wherein C and G are unmethylated, X₁, X₂, X₃ and X₄ are nucleotides and a GCG trinucleotide sequence is not present at or near the 5′ and 3′ termini. (See U.S. Pat. No. 6,194,388, which is hereby incorporated by reference in its entirety.)

Preferably, oligodeoxyribonucleotides (ODNs) for use with the disclosed invention are in the range of about 20-24 nucleotides length, although ODN sequences with as few as 6 nucleotides have been reported to be effective also (Wang, S. et al, Vaccine (2003) 21:4297-4306). Each one contains a “CpG” sequence in the middle of the ODN. These dinucleotide sequences are unmethylated, thus mimicking those nucleotides found in bacterial DNA, in contrast to vertebrate DNA, in which these sequences are methylated (and underrepresented, i.e., suppressed).

Some examples of ODNs are listed below:

| | | | |
|---|---|---|---|
| CpG ODN 1826: | TCCATGACGTTCCTGACGTT; | (SEQ ID NO: 1) | |
| CpG ODN 1760: | ATAATCGACGTTCAAGCAAG; | (SEQ ID NO: 2) | |
| non-CpG ODN 1908: | ATAATAGAGCTTCAAGCAAG; | (SEQ ID NO: 3) | |
| non-CpG ODN 1745: | TCCAATGAGCTTCCTGAGTCT; | (SEQ ID NO: 4) | |
| hexamer CpG: | GACGTT; | (SEQ ID NO: 5) | |
| D-ODN D35: | GGTGCATCGATGCAGGGGGG; | (SEQ ID NO: 6) | |
| D-ODN 2216: | GGTGCATCGATGCAGGGGGG; | (SEQ ID NO: 7) | |
| K-ODN DSP30: | TCGTCGCTGTCTCCGCTTCTTCTTGCC; | (SEQ ID NO: 8) | |
| K-ODN 2006: | TCGTCGTTTTGTCGTTTTGTCGTT; | (SEQ ID NO: 9) | |
| K-ODN K3: | ATCGACTCTCGAGCGTTCTC; | (SEQ ID NO: 10) | |
| K-ODN K23: | TCGAGCGTTCTC; | (SEQ ID NO: 11) | and |
| K-ODN ISS: | TGACTGTGAACGTTCGAGATGA. | (SEQ ID NO: 12) | |

(A=adenosine, C=cytidine, G=guanosine, T=thymidine).

In an alternative embodiment, cytosine-guanosine-independent ODNs (non-CpG ODNs) may be used as adjuvants with the disclosed methods. Non-CpG ODNs typically comprise the general formula PyNTTTTGT in which Py is C or T, and N is A, T, C, or G. (Elias, et al., J. Immun. (2003) 171:3697-3704.) Non-CpG ODNs may be used alone or with other adjuvants and may also be used with CpG ODNs.

### Administration and use

The described invention thus concerns and provides a means for preventing or attenuating infection by Flavivirus. As used herein, a vaccine is said to prevent or attenuate a disease if its administration to an individual results either in the total or partial immunity of the individual to the disease, or in the total or partial attenuation (i.e. suppression) of a symptom or condition of the disease.

A composition is said to be “pharmacologically acceptable” if its administration can be tolerated by a recipient patient. Such an agent is said to be administered in a “therapeutically effective amount” if the amount administered is physiologically significant. An agent is physiologically significant if its presence results in a detectable change in the physiology of a recipient patient.

The active vaccines of the invention can be used alone or in combination with other active vaccines such as those containing other active subunits to the extent that they become available. Corresponding or different subunits from one or several serotypes may be included in a particular formulation.

The therapeutic compositions of the described invention can be administered parenterally by subcutaneous or intramuscular injection.

Many different techniques exist for the timing of the immunizations when a multiple administration regimen is utilized. It is possible to use the compositions of the invention more than once to increase the levels and diversities of expression of the immunoglobulin repertoire expressed by the immunized animal. Typically, if multiple immunizations are given, they will be given one to two months apart.

To immunize subjects against dengue fever, for example, the vaccines containing the subunits are administered to the subject in conventional immunization protocols involving, usually, multiple administrations of the vaccine. Administration is typically by injection, typically intramuscular or subcutaneous injection; however, other systemic modes of administration may also be employed.

According to the described invention, an “effective amount” of a therapeutic composition is one which is sufficient to achieve a desired biological effect. Generally, the dosage needed to provide an effective amount of the composition will vary depending upon such factors as the animal's or human's age, condition, sex, and extent of disease, if any, and other variables which can be adjusted by one of ordinary skill in the art. The antigenic preparations of the invention can be administered by either single or multiple dosages of an effective amount. Effective amounts of the compositions of the invention can vary from 0.01-100 μg per dose, more preferably from 0.1-20 μg per dose, and most preferably 1-5 μg per dose.

### EXAMPLES

The examples below demonstrate that the ability of a particular adjuvant to enhance the immunogenicity of the dengue subunit vaccine is extremely variable and cannot be predicted a priori. The data in Table 2 below indicate that the immune response to the vaccine formulated with different adjuvant combinations can vary from highly vigorous to undetectable. Thus, the selection of an effective vaccine formulation must be determined experimentally. Hence, the invention described herein is unique in its immunogenic properties. Furthermore, in addition to the antibody response (“Th2” type immune response) reported in Table 2, the examples demonstrate that the tetravalent dengue recombinant subunit vaccine formulated with the adjuvant combination of QS-21+CpG elicits a robust cell-mediated (“Th1” type) immune response as indicated by antigen-stimulated production of higher levels of IFN-γ from immune splenocytes in vitro than with vaccines formulated with the other adjuvant combinations tested. In addition, viral neutralizing antibody titers produced by this vaccine formulation (QS-21+CpG) were comparable to all other vaccine/adjuvant combinations tested. Moreover, the examples show that the addition of small amounts of NS1 to an 80% E vaccine dramatically increases the amount of IFN-γ produced by immune splenocytes in vitro upon antigenic stimulation. This result supports the inclusion of NS1 in the active ingredients of the vaccine as providing additional benefit to the vaccine. Finally, it is well documented that particular CpGs are much more effective stimulators of immune responses in one species relative to another species. Therefore, it is critical to distinguish among these different CpGs and choose the appropriate one to obtain the optimal immune response in the host species (humans). The examples provided below demonstrate this distinction among CpGs, and the choice of the appropriate CpG for inclusion in the vaccine formulation.

The following examples are intended to illustrate but not to limit the invention.

### Example 1

### Combinations of the Dengue Recombinant Subunit Vaccine with Various Adjuvants Engender Widely Varying Immune Responses

Balb/c mice were given two subcutaneous injections of tetravalent dengue vaccine (2.5 μg of each serotype 80% E+0.5 μg of NS1), 4 weeks apart, using a variety of adjuvants. The tetravalent vaccine comprises a ZipperII of DEN-4 80% E and monomeric 80% E from DEN1, DEN2 and DEN3. Mice were exsanguinated 14 days post booster vaccination, and sera collected from mice within each group were pooled. The sera were then titrated for the ability to neutralize dengue virus using an in vitro assay, the “plaque reduction neutralization test” (PRNT; Russell, P. K., and Nisalak, A. A., J. Immunol. (1967) 99:285-290). Results for PRNT titers against dengue serotype 2 are shown in Table 2 below.

TABLE 2
PRNT titers to dengue serotype 2 elicited in mice vaccinated
with the dengue recombinant subunit vaccine

| Adjuvant | PRNT titer^{a} | Adjuvant | PRNT titer^{a} | Adjuvant | PRNT titer^{a} |
|---|---|---|---|---|---|
| QS-21 | 210 | RC529^{b} | <10 | CpG | 50 |
| QS-21 + RC529 | <10 | RC529 + CpG | <10 | CpG + ISA51^{c} | <10 |
| QS-21 + CpG | 350 | RC529 + ISA51 | <10 | CpG + ISA720^{d} | 15 |
| QS-21 + ISA51 | 360 | RC529 + ISA720 | 60 | CpG + alum^{e} | <10 |
| QS-21 + ISA720 | 580 | RC529 + alum | <10 | CpG + ChitoZN^{f} | 130 |
| QS-21 + alum | 120 | RC529 + ChitoZN | 10 | alum | 100 |
| QS-21 + ChitoZN | <10 | ISA720 | 125 | ChitoZN | 25 |
| ISA51 | 100 | | | | |

^{a}Highest dilution of serum capable of neutralizing 50% of dengue virus in the PRNT assay.
^{b}A “monophosphoryl lipid A-like” adjuvant obtained from Corixa Corp., Hamilton, MT.
^{c}A mineral oil similar to incomplete Freund's adjuvant obtained from Seppic, Inc., Fairfield, NJ.
^{d}A biodegradable oil obtained from Seppic, Inc., Fairfield, NJ.
^{e}Aluminum hydroxide/magnesium hydroxide gel, obtained preformulated from Pierce Chemical Co., Rockford, IL.
^{f}An insoluble microparticulate complex of chitosan and zinc which binds antigens through ionic interactions, obtained from Zonagen, Inc., The Woodlands, TX.

The results shown in Table 2 above demonstrate that the ability of a particular adjuvant to enhance the immunogenicity of the dengue subunit vaccine is extremely variable and is difficult to predict accurately a priori. Thus, the selection of an effective vaccine formulation is unexpected, and must be determined experimentally. Hence, the invention described herein is unique in its immunogenic properties.

### Example 2

### Saponin and ODN Elicits Higher Cell Mediated Response than Other Combinations

Balb/c mice were given two subcutaneous injections of tetravalent dengue vaccine (3 μg of each serotype 80% E, +0.3 μg of NS1), 4 weeks apart. Four days and seven days post booster vaccination, two mice from each vaccinee group were sacrificed and splenectomies performed. Spleen cells (pooled from the two mice) were cultured in vitro with each dengue antigen as stimulant for 5 days. Culture supernatants were then harvested and analyzed for IL-4 and IFN-γ by an ELISA technique (Katial, R. K. et al., Clin. Diagn. Lab. Immunol. (1998) 5:78-81), using antibodies specific for mouse cytokines. Splenocyte cultures stimulated with each antigen for 7 days were also assayed for proliferative capacity by tritiated thymidine incorporation (Katial, R. K. et al., J. Clin. Immunol. (1997) 17:34-42), using a 96-well microplate format. Fourteen days post booster vaccination, the remaining mice in each group were exsanguinated, and sera from mice within each group were pooled and analyzed by the PRNT assay described above. The results are given in Table 3 below.

TABLE 3
Immune Response to Dengue Vaccine with Adjuvant Combinations

| Dengue | | | | | | | Negative |
|---|---|---|---|---|---|---|---|
| serotype | Assay | QS-21 | QS-21 + CpG | QS-21 + ISA51^{a} | QS-21 + ISA720^{b} | CpG + ChitoZN^{c} | Control^{d} |
| D1 | PRNT titer^{e} | 830 | 420 | 2040 | 1040 | 350 | <10 |
| | antigen binding | 5800 | 11,400 | 16,700 | 6800 | 7000 | <100 |
| | antibody^{f} | | | | | | |
| | IL-4^{g} | 0.52 | 0.29 | 0.41 | 0.43 | 0.23 | 0.28 |
| | IFN-γ^{h} | 2.84 | 4.80 | 2.62 | 2.32 | 1.52 | 0.63 |
| | lymphocyte | 46,052/ | 18,674/ | 34,888/ | 38,848/ | 27,962/ | 544/ |
| | proliferation (net | 19.0 | 10.3 | 15.4 | 33.5 | 20.8 | 1.2 |
| | cpm)/(SI)ⁱ | | | | | | |
| D2 | PRNT titer^{e} | 900 | 3030 | 3330 | 1000 | 3800 | <10 |
| | antigen binding | 6900 | 16,500 | 20,200 | 7500 | 10,400 | <100 |
| | antibody^{f} | | | | | | |
| | IL-4^{g} | 0.54 | 0.32 | 0.54 | 0.52 | 0.36 | 0.38 |
| | IFN-γ^{h} | 4.47 | 14.68 | 5.66 | 4.31 | 4.17 | 0.76 |
| | lymphocyte | 62,289/ | 31,538/ | 39,843/ | 22,962/ | 14,811/ | 0/ |
| | proliferation (net | 24.7 | 10.1 | 16.6 | 14.0 | 11.1 | 0.7 |
| | cpm)/(SI)ⁱ | | | | | | |
| D3 | PRNT titer^{e} | 120 | 70 | 230 | 190 | 260 | <10 |
| | antigen binding | 7800 | 23,300 | 23,300 | 7600 | 13,300 | <100 |
| | antibody^{f} | | | | | | |
| | IL-4^{g} | 1.16 | 0.64 | 1.18 | 0.78 | 0.50 | 0.36 |
| | IFN-γ^{h} | 5.52 | 15.45 | 4.67 | 3.12 | 3.94 | 1.04 |
| | lymphocyte | 47,212/ | 30,404/ | 43,743/ | 29,575/ | 18,498/ | 0/ |
| | proliferation | 13.2 | 13.4 | 19.0 | 24.1 | 13.7 | 0.7 |
| | (net cpm)/(SI)ⁱ | | | | | | |
| D4 | PRNT titer^{e} | 90 | 320 | 1430 | 60 | 170 | <10 |
| | antigen binding | 10,600 | 31,000 | 39,800 | 8600 | 22,400 | <100 |
| | antibody^{f} | | | | | | |
| | IL-4^{g} | 1.30 | 0.42 | 0.82 | 0.53 | 0.42 | 0.44 |
| | IFN-γ^{h} | 12.17 | 16.80 | 12.51 | 7.68 | 7.46 | 1.42 |
| | lymphocyte | 59,122/ | 28,356/ | 24,268/ | 22,576/ | 24,099/ | 285/ |
| | proliferation (net | 16.0 | 9.8 | 10.6 | 20.7 | 18.2 | 1.2 |
| | cpm)/(SI)ⁱ | | | | | | |

^{a}A mineral oil similar to incomplete Freund's adjuvant obtained from Seppic, Inc., Fairfield, NJ.
^{b}A biodegradable oil obtained from Seppic, Inc., Fairfield, NJ.
^{c}An insoluble microparticulate complex of chitosan and zinc which binds antigens through ionic interactions, obtained from Zonagen, Inc., The Woodlands, TX.
^{d}The negative control consisted of a combination of QS-21 + ISA720 without the Dengue antigens.
^{e}Titer of serum viral neutralizing antibodies determined as the highest dilution of serum capable of neutralizing 50% of Dengue virus of a given serotype in an in vitro assay (“plaque reduction neutralization test”).
^{f}Titer of antigen binding antibodies in serum determined in an ELISA format as the highest dilution of serum yielding 50% of maximal antigen binding.
^{g}Concentration of IL-4 (ng/ml) in supernatants from splenocyte cultures (at 2 × 10⁶ cells/ml) after 5 days of incubation. Results given are the average of the data obtained from the 4 days and 7 days post booster vaccination cell populations.
^{h}Concentration of IFN-γ (ng/ml) in supernatants from splenocyte cultures (at 2 × 10⁶ cells/ml) after 5 days of incubation. Results given are the average of the data obtained from the 4 days and 7 days post booster vaccination cell populations.
ⁱNet cpm = cpm from antigen stimulated cultures (average of quadruplicates) minus cpm from unstimulated cultures (average of quadruplicates). SI (stimulation index) = (cpm from stimulated cultures minus cpm from medium controls)/(cpm from unstimulated cultures minus cpm from medium controls). Cultures contained 4 × 10⁵ cells/0.2 ml. Results given are the average of the data obtained from the 4 days and 7 days post booster vaccination cell populations.

The results given in Table 3 above demonstrate that the tetravalent dengue recombinant subunit vaccine formulated with the adjuvant combination of QS-21+CpG elicits a robust cell-mediated immune response as indicated by antigen-stimulated production of higher levels of IFN-γ (see bold portion of Table) from immune splenocytes in vitro than with vaccines formulated with the other adjuvant combinations tested. In addition, viral neutralizing antibody titers produced by this vaccine formulation (QS-21+CpG) were comparable to all other vaccine/adjuvant combinations tested and is superior to that of QS21 alone which also provide strong responses.

### Example 3

### The Addition of NS1 to a Dengue 80% E Subunit Vaccine Increases the Cell-Mediated Immune Response

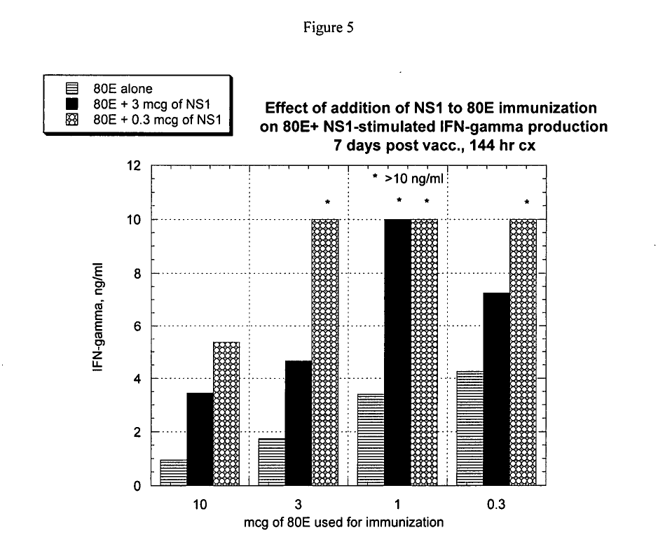

Balb/c mice were vaccinated with combinations of 80% E and NS1 from dengue serotype 2 using varying doses of both proteins. Seven days post booster vaccination, splenectomies were performed and splenocyte cultures established. The 80% E and NS1 proteins (each at a final concentration of 5 μg/ml) were used as stimulants in the splenocyte cultures. After 6 days of culture, samples of culture supernatants were collected and analyzed for interferon-γ production by ELISA. Results are depicted in FIG. 5 below. The results shown in FIG. 5 demonstrate that the addition of as little as 0.3 μg of NS1 to an 80% E vaccine, varying in dose from 0.3 to 10 μg of 80% E, dramatically increases the amount of IFN-γ produced by immune splenocytes in vitro upon antigenic stimulation. As discussed above, IFN-γ is a cytokine produced by “Th1” type T helper lymphocytes, which mediate cellular immunity, i.e., the activation of functional effector cells, such as cytotoxic T lymphocytes (CTL).

### Example 4

### CpG Adjuvants are Species-Specific and the Choice of the Appropriate CpG is Important for Desired Effects

It is well documented that particular CpGs are much more effective stimulators of immune responses in one species relative to another species. In order to determine the relative stimulatory capabilities of different CpGs for human peripheral blood mononuclear cells (PBMC) and to confirm their relative species specificity, the following experiment was performed.

A. Stimulation of Human PBMC by CpGs

1) Human PBMC were prepared from heparinized whole blood specimens by Ficoll-hypaque centrifugation.

2) Separated PBMC were washed, resuspended, and cultured in medium containing CpGs at concentrations varying within the range of 0.16 to 5 μg/ml.

3) Cultures were assayed for proliferative capacity by tritiated thymidine uptake.

4) At an appropriate time, culture supernatants were assayed for production of immunoglobulins.

B. Stimulation of Murine Splenocytes by CpGs

1) Naïve Balb/c mice were sacrificed and splenectomies performed.

2) Splenocyte suspensions were prepared and splenocytes cultured in medium containing the same CpGs at the same concentrations as above (0.16 to 5 μg/ml).

3) Cultures were assayed for proliferative capacity by tritiated thymidine uptake.

4) At an appropriate time, culture supernatants were assayed for production of particular cytokines.

The results are summarized in Table 4 below.

TABLE 4
Comparison of CpGs for Lymphocyte Stimulation Activity

| Subjects | | | | | | | | | |
|---|---|---|---|---|---|---|---|---|---|
| (Human PBMC | Stimulation Index^{a} | | | IgG^{b} | | | IgM^{c} | | |
| and Balb/C | ODN | ODN | | ODN | ODN | | ODN | ODN | |
| splenocytes) | 10103 | 2137 | CpG-A | 10103 | 2137 | CpG-A | 10103 | 2137 | CpG-A |
| RORO (human) | 13.6 | 4.5 | 2.2 | 1.80 | 0.33 | 0.95 | 208 | 35 | 13 |
| EAG (human) | 11.0 | 3.7 | 2.1 | 0.50 | 0.02 | 0.02 | 129 | 11 | 3 |
| GDRN (human) | 48.4 | 12.0 | 5.1 | ND^{d} | ND | ND | ND | ND | ND |
| RDRL (human) | 25.6 | 11.0 | 6.4 | ND | ND | ND | ND | ND | ND |
| Naive Balb/C | 14.3 | 2.5 | 94.1 | ND | ND | ND | ND | ND | ND |
| (Splenocytes | | | | | | | | | |

^{a}Stimulation index calculated for lymphocyte proliferation assays stimulated with each of the CpGs at 0.31 μg/ml for the human subjects and 1.25 μg/ml for mice.
^{b}IgG (μg/ml) produced by lymphocyte cultures stimulated with each of the CpGs at 0.31 μg/ml.
^{c}IgM (ng/ml) produced by lymphocyte cultures stimulated with each of the CpGs at 0.31 μg/ml.
^{d}not done

The results shown in Table 4 above indicate that ODN 10103 is far superior to either of the other two CpGs tested for stimulation of human PBMC in vitro as determined by lymphocyte proliferation assays as well as immunoglobulin synthesis (both IgG and IgM). However, CpG-A is far superior to either of the other two CpGs tested (ODN 10103 included) for stimulation of murine splenocytes in vitro as determined by lymphocyte proliferation assays (and IL-10 production; data not shown). Thus, the species specificity of these CpGs was confirmed by these experiments, and the choice of the appropriate CpG (ODN 10103) for inclusion in a vaccine formulation was made based on the results of these experiments.

### Example 5

### Expression of West Nile Proteins in the Drosophila S2 System

The expression plasmid pMttbns (derived from pMttPA) contains the following elements: Drosophila melanogaster metallothionein promoter, the human tissue plasminogen activator secretion leader (tPAL) and the SV40 early polyadenylation signal. At Hawaii Biotech a 14 base pair BamHI fragment was excised from the pMttbns vector to yield pMttΔXho that contains a unique XhoI site in addition to an existing unique BglII site. This expression vector targets expressed proteins to be secreted into the culture medium. All West Nile sequences were introduced into the pMttΔXho vector using these unique BglII and XhoI sites. For the expression of a carboxy-truncated West Nile envelope protein, a synthetic gene encoding the prM protein and 80% of the E protein from West Nile virus was synthesized (Midland Certified Reagent Co., Midland, Tex.). The nucleotide sequence of the synthetic gene follows the published sequences of West Nile viruses isolated in 1999 in New York City (23). The C-terminal truncation of the E protein at amino acid 401 eliminates the transmembrane domain of the E protein (in a fashion analogous to Hawaii Biotech's dengue envelope protein vaccines), and therefore can be secreted into the medium. For the expression of a full-length West Nile NS1 protein a gene fragment was generated by RT-PCR. The NS1 gene fragment represents nucleotides 2470 to 3525 on the genome and codes for a product containing 352 amino acid residues. Both the synthetic prM80E and RT-PCR generated NS1 gene fragments were designed to include restriction endonuclease sites that were used for cloning and also included two stop codons immediately following the last West Nile codon. The final prM80E plasmid construct was designated pMttWNprM80E and the NS1 plasmid construct was designated pMttWNNS1.

S2 cells were co-transformed with the pMttΔXho-based expression plasmids and the pCoHygro selection plasmid that encodes hygromycin resistance utilizing the calcium phosphate co-precipitation method or with Cellfectin (Invitrogen Kits, Carlsbad, Calif.) according to the manufacturer's recommendations. Cells were co-transformed with 20 μg total DNA with a 20:1 ratio of expression plasmid to selection plasmid. Transformants were selected with hygromycin B (Roche Molecular Biochemicals, Indianapolis, Ind.) at 300 μg/ml. Following selection, cells were adapted to growth in the serum free medium Excel 420 (JRH, Lenexa, Kans.). For expression studies, cells were grown in Excel 420, 300 μg/ml hygromycin, and induced with 200 μM CuSO4. Cells were seeded at a density of 2×10⁶ cells/ml and allowed to grow for 6-7 days. Under optimal conditions, cell densities of 1.5 to 2×10⁷ cells/ml were achieved after 6-7 days of growth. The culture supernatant was examined for expressed protein by SDS-PAGE and Western blot.

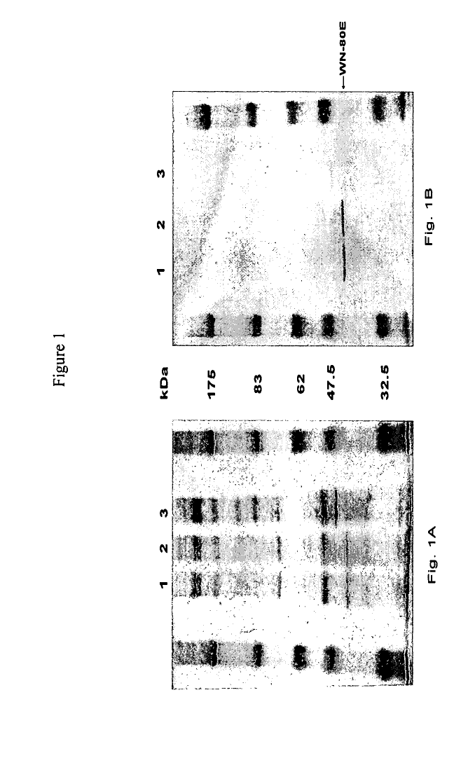

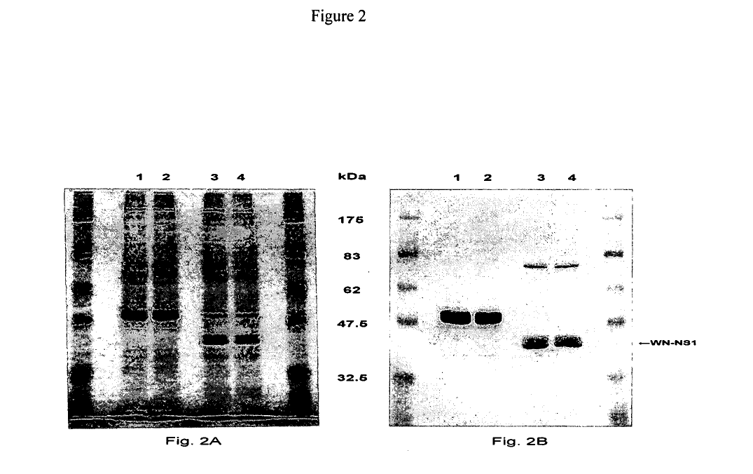

For the detection of West Nile 80E on Western blots a rabbit polyclonal anti-West Nile virus antibody (BioReliance Corp.) followed by an anti-rabbit IgG-alkaline phosphatase conjugated secondary antibody was used. For the detection of West Nile NS1 on Western blots the flavivirus group specific anti-NS1 monoclonal 7E11 followed by an anti-mouse IgG-alkaline phosphatase conjugated secondary antibody was used. The blots were developed with NBT/BCIP (Sigma Chem. Co.) solid phase alkaline phosphatase substrate. Results are shown in FIGS. 1A and 1B and 2A and 2B.

### Example 6

### Purification of West Nile 80E and NS1

Purification protocols were developed for both the West Nile envelope protein (80E) and non-structural protein 1 (NS1). The procedures are based upon existing methods that are currently utilize for manufacturing of dengue antigens for in vitro diagnostic use and intend to utilize for the manufacture of a dengue vaccine. Purification of both proteins was accomplished by immunoaffinity chromatography (LAC). For 80E, the monoclonal antibody (MAb) 4G2 was utilized, while the monoclonal antibody 7E11 was utilized for purification of NS1. Briefly, the procedure involves filtration of the medium using a Whatman 1 filter. The crude material is then loaded onto the IAC column, which contains immobilized MAb that is covalently coupled via N-hydroxysuccinimide chemistry, at a linear flowrate of 2 cm/min for 80E and 1.2 cm/min for NS1. After the sample is loaded, the matrix is washed with 10 mM phosphate buffered saline, pH 7.2, containing 0.05% (v/v) tween-20 (PBST, 140 mM NaCl). Bound protein is eluted from the IAC column with 20 mM glycine buffer, pH 2.5. The eluent is neutralized then buffer exchanged against either PBST (for 80E) or 10 mM PB (for NS1). The purification products are routinely analyzed by sodium dodecyl sulfate polyacrylamide gel electrophoresis (SDS-PAGE) with Coomassie or silver staining, Western blot, UV absorption, and sandwich ELISA to determine purity, identity, quantity, and bioactivity. In addition, samples were analyzed by N-terminal amino acid sequencing and amino acid analysis. These analyses provided confirmation of identity and quantity of the purification products. Yields from the columns have proven to be consistent for both proteins with satisfactory recoveries, thus indicating that, if used in the current formats, these processes should be applicable to product manufacture.

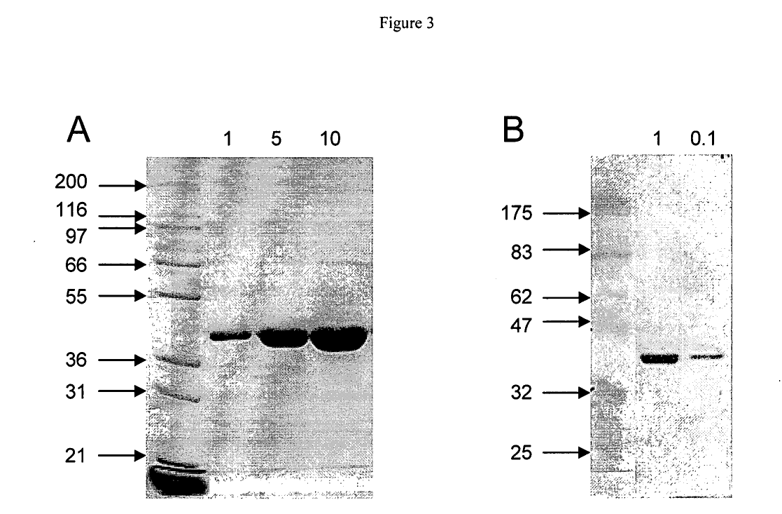

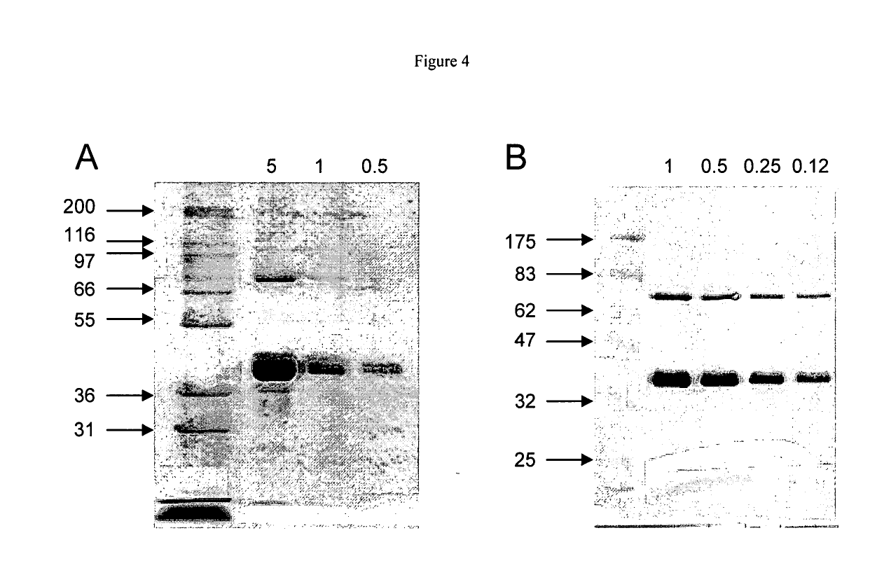

Representative SDS-PAGE and Western blot profiles of the two purified proteins are presented in FIGS. 3 and 4. For the analysis, both samples were run under non-reducing conditions. The 80E molecule migrates as a single band with a relative molecular weight consistent with that determined from the amino acid composition (i.e., 43 kD). This finding indicates that disulfide bond formation is not occurring between molecules, although aggregates (e.g. -dimers) stabilized by noncovalent interactions could still be present in the native state. Trace contaminants (˜5 bands) are visible in a 10 μg load on the Coomassie stained gel. Assuming a threshold of detection of 100 ng, the purity of the 80E can be estimated at >90%. In contrast, the NS1 migrates as two distinct forms: one with a relative molecular weight that is consistent with that expected for a monomeric form (40 kD) and one with a relative molecular weight that is consistent with a dimeric form (80 kD). Unlike the 80E preparation, a major contaminant is clearly visible in a 5 μg load with possibly 2-3 minor contaminants as well. As the major contaminant is still visible in a 1 μg load but not a 0.5 μg load, the purity of the NS1 preparations are estimated at 90%.

### Immune Response of West Nile Vaccine Formulations in Mice

The cellular and humoral immunogenicity of the purified recombinant subunit vaccine was evaluated in mice. Balb/c mice (8 weeks old) were vaccinated twice, subcutaneously, with a 4 week interval with the indicated amounts (see below) of antigens plus adjuvant. Seven days post booster vaccination, splenectomies were performed on 2 mice from each group and splenocyte suspensions prepared. Erythrocytes were lysed with an NH₄Cl based lysis solution, and the cell pellet resuspended in cell culture medium. Cell counts were performed on each suspension using a hemacytometer, and the suspensions diluted to 4×10⁶ cells/ml for lymphocyte proliferation and cytokine production assays.

### Example 7

### Lymphocyte Proliferation Assays

Aliquots (0.1 ml) of each splenocyte suspension were dispensed into wells of a 96-well cell culture plate. Aliquots (0.1 ml) of the West Nile antigens (80% E and/or NS1) were then dispensed into the wells containing each of the cell suspensions (in quadruplicate), at a final concentration of 5 μg/ml of each antigen. Wells with unstimulated (antigen omitted) cell suspensions, as well as phytohemagglutinin (PHA) stimulated cell suspensions (as a positive control) were also included. Cultures were incubated at 37° C./5% CO₂/humidified for 7 days (3 days for PHA stimulated cultures), and then one microcurie of tritiated (methyl-³H) thymidine (60 Ci/mmol; ICN Biomedicals, Inc., Irvine, Calif.) was added to each well (in a volume of 0.01 ml), and incubation continued for 18 hrs. After that period of time, the cell cultures were harvested onto glass fiber filtration plates and washed extensively using a vacuum driven harvester system (Filtermate Plate Harvester, Packard Instrument Co.). The filtration plates were then analyzed for radioactivity using the TopCount Microplate Scintillation and Luminescence Counter (Packard Instrument Co.).

### Example 8

### Cytokine Production Assays

Aliquots (0.5 ml) of each splenocyte suspension were dispensed into wells of a 24-well cell culture plate. Aliquots (0.5 ml) of the same antigens used for lymphocyte proliferation were dispensed into the wells containing each of the cell suspensions. Unstimulated and pokeweed mitogen (PWM)-stimulated cell suspensions were also included. Cultures were incubated for 5 days at 37° C./5% CO₂/humidified. The culture supernatants were then harvested and frozen prior to analysis for specific cytokines. The cytokines interferon-gamma (IFN-γ), interleukin-4 (IL-4), and IL-10 were assayed by a standard enzyme-linked immunosorbent assay (ELISA) technique.

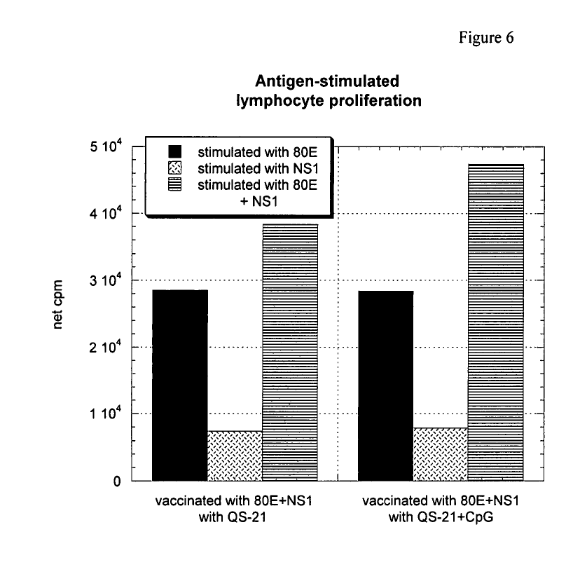

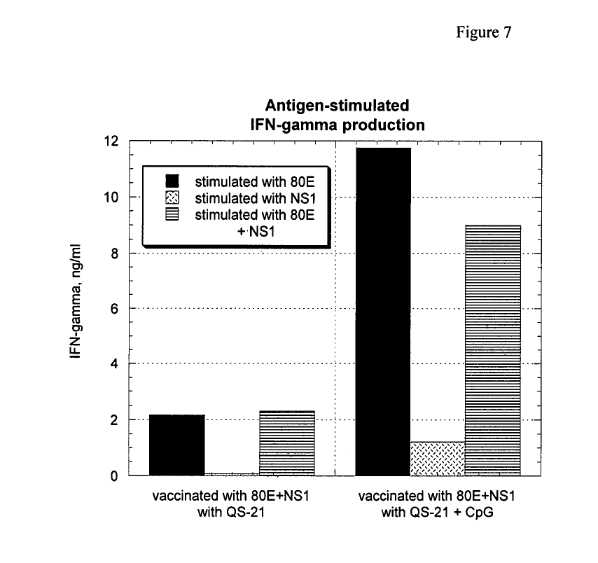

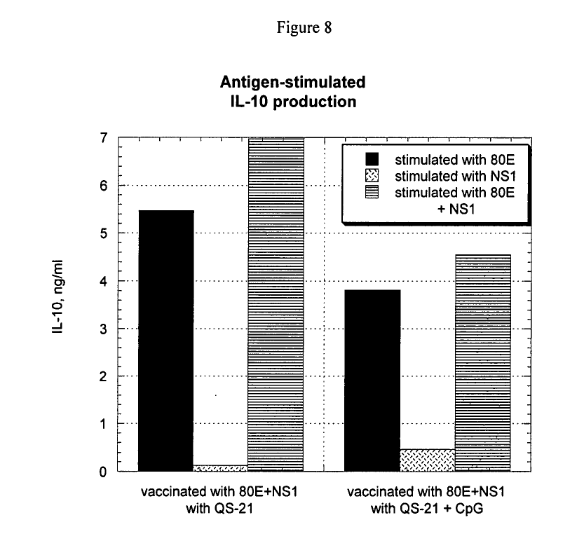

The results of these cellular immunity assays are presented in FIGS. 6, 7 and 8. Splenocytes from mice immunized with 3 μg of 80E+0.3 μg of NS1, cultured in vitro with vaccine antigens, showed excellent proliferation (FIG. 6), IFN-γ production (FIG. 7), and IL-10 production (FIG. 8). Production of IL-4 was similar to IL-10 (data not shown). The level of antigen-stimulated lymphocyte proliferation and cytokine production was comparable to the level of mitogen (phytohemagglutinin or pokeweed mitogen) driven lymphocyte proliferation or cytokine production (data not shown). Thus, the recombinant subunit vaccine produces a robust cellular immune response in mice, as demonstrated by antigenic stimulation in vitro.

### Example 9

### Antibody Response

In addition, the antibody response to vaccination was determined on serum samples collected from individual mice 14 days post booster vaccination. Sera were titrated for antibodies to both the 80E and NS1 proteins by a standard ELISA technique using plates coated with the individual antigens. The results of these assays are given in Table 5, and demonstrate that the vaccine elicits a high titer antibody response in mice to 80E at either a 3 or 10 μg immunizing dose (titer>1:10,000). At a 0.3 or 1 μg immunizing dose of NS1, titers were between 1:1000 and 1:10,000.

TABLE 5
Mouse Immunogenicity Experiment Antibody Assays^{a}

Antibody Titer^{b} to

| Group no. | 80E | NS1 |
|---|---|---|
| 1 | 15,700^{c} | —^{d} |
| | (9650-25,800) | |
| 2 | 26,400 | 2030 |
| | (10,900-64,000) | (670-6180) |
| 3 | 19,900 | 2160 |
| | (7300-54,200) | (950-4940) |
| 4 | 20,500 | 3160 |
| | (12,200-34,300) | (1860-5400) |
| 5 | 18,100 | 5100 |
| | (14,200-23,100) | (3150-8200) |
| 6 | 15,600 | 5320 |
| | (5700-42,800) | (1720-16,350) |
| 7 | 14,300 | 3670 |
| | (6500-31,400) | (1820-7420) |

^{a}Groups of mice were immunized, subcutaneously, twice at a 4 week interval with the following antigen/adjuvant combinations:
1. 3 μg 80E in PBS/tween + 0.3 μg NS1/QS-21
2. 3 μg 80E in PBS + 0.3 μg NS1/QS-21
3. 3 μg 80E in PBS/tween + 0.3 μg NS1/QS-21 + ISS1018
4. 3 μg 80E in PBS + 0.3 μg NS1/QS-21 + ISS1018
5. 10 μg 80E in PBS/tween + 1 μg NS1/QS-21
6. 10 μg 80E in PBS + 1 μg NS1/QS-21
7. 3 μg 80E in PBS/tween + 0.3 μg NS1/ISCOMATRIX Mice were exsanguinated 14 days post booster vaccination, and the sera analyzed for antibody titers.
^{b}ELISA antibody titer (dilution of serum yielding 50% maximum absorbance value; wells coated with either 80E prepared in PBS/tween or NS1
^{c}GMT; (range, +/− standard deviation); n = 5 for groups 1-4; n = 4 for groups 5-7
^{d}data presently unavailable for NS1 assay for group 1

### Example 10

### Protective Efficacy of West Nile Vaccine Formulations in the Golden Hamster Model

The protective efficacy of the vaccine was evaluated in the golden hamster model of West Nile encephalitis (Xiao, S-Y et al., Emerg. Infect. Dis. 7:714-721, 2001; Tesh, R. B. et al., Emerg. Infect. Dis. 8:245-251, 2002). Formulated vaccines (purified E+/−NS1 proteins mixed with adjuvants) were sent to Dr. Robert Tesh at UT Medical Branch, Galveston. The experimental protocol consisted of the following steps:

1) 30 hamsters were immunized, subcutaneously, with each particular vaccine formulation (specific dose of immunogens and adjuvant). A control group of 30 hamsters was administered adjuvant only. One group of hamsters received a vaccine formulation in which NS1 was omitted.

2) Hamsters were given a booster immunization at 33 days.

3) 17 days after the booster vaccination, 12 hamsters from each group were bled and antibody titers to West Nile virus determined by hemagglutination inhibition, complement fixation, and PRNT assays.

4) Immediately after the blood samples were obtained, all hamsters were challenged by administration of 10⁴ 50% tissue culture infective doses (TCID₅₀) of live virus.

5) Hamsters were then bled daily for 6 days following challenge to determine the level of viremia and the antibody response to viral challenge.

6) Animals were held for 30 days following challenge for observation of morbidity and mortality. (Using this protocol, about 50% of the challenged animals die, usually between the 7^{th} and 14^{th} days following challenge.)

7) At the end of the 30 day holding period, the surviving animals were bled once more for antibody determinations, and then euthanized.

These results are given in Table 6 below, and demonstrate complete protection (100% survival) of vaccinated hamsters challenged with a lethal dose of live virus. The vaccinated and challenged animals also showed no evidence of clinical disease. The p value calculated by the Fisher exact probability test for either vaccinated group relative to the adjuvant control group (23% survival) was <0.00001. Thus, the recombinant subunit vaccine provides solid protection in a clinically relevant animal model of West Nile disease. In addition, hemagglutination inhibition (HI), complement fixation (CF), and viral neutralizing (“plaque reduction neutralization test”; PRNT) antibody titers were measured as well as determinations of viremia performed. The results of the antibody assays are presented in Tables 7 and 8, and indicate that an excellent immune response to the vaccine was developed in hamsters. The antibody titers generated were as high or higher than what had been seen with a live virus vaccine in this model (Tesh, R. B. et al., Emerg. Infect. Dis. 8:1392-1397, 2002). Moreover, the HI antibody titers obtained in the pre-challenge sera (day 0 post challenge) were as high as day 6 post challenge (when antibody titers in the adjuvant control group became evident; Table 8. This is considered to be good evidence that no viral replication occurred in the vaccinated animals upon challenge, i.e., the vaccine engendered “sterile immunity”. Results of the viremia determinations support this conclusion. No viremia was detectable in any of the immunized animals. In contrast, the pattern of viremia observed in the adjuvant control group animals (Table 9) was similar to that seen in naive adult hamsters receiving the same virus dose (Xiao, S-Y, vide supra). Thus, the recombinant subunit vaccine is at least equivalent in potency to the live, attenuated, chimeric vaccine in the golden hamster model of West Nile virus encephalitis.

TABLE 6
Protective Efficacy in Golden Hamsters^{a}

| | | # survivors/ | |
|---|---|---|---|
| Group | Immunogen | total challenged | % survival |
| 1 | E protein (10 μg) | 30/30^{b} | 100 |
| 2 | E protein (10 μg) + NS-1 | 30/30^{b} | 100 |
| | protein (1 μg) | | |
| 3 | none (adjuvant only) | 7/30 | 23 |

^{a}animals were vaccinated twice, subcutaneously, with a 1 month interval using the indicated amounts of proteins plus adjuvant (or adjuvant alone); animals were then challenged 17 days post booster vaccination with 10⁴ TCID₅₀ doses of live virus
^{b}p value < 0.00001 by Fisher exact probability test relative to adjuvant control group

TABLE 7
Results of hemagglutination-inhibition (HI), complement-fixation
(CF) and plaque reduction neutralization (PRNT) tests done
on sera of hamsters after two immunizations with
West Nile recombinant subunit vaccines
(Hamsters bled 17 days after second immunization)

Antibody Titer

| ANIMAL NUMBER | HI | CF | PRNT |
|---|---|---|---|
| Group 1 | | | |
| 2901 | 1:320 | 1:320 | 1:1280 |
| 2902 | 1:160 | 1:160 | 1:640 |
| 2903 | 1:160 | 1:160 | 1:1280 |
| 2904 | 1:160 | 1:80 | 1:640 |
| 2905 | 1:320 | 1:160 | 1:640 |
| 2906 | 1:640 | 1:320 | 1:640 |
| 2907 | 1:640 | 1:320 | 1:1280 |
| 2908 | 1:320 | 1:160 | 1:2560 |
| 2909 | 1:320 | 1:160 | 1:640 |
| 2910 | 1:640 | 1:320 | 1:1280 |
| 2911 | 1:640 | 1:320 | 1:1280 |
| 2912 | 1:640 | 1:320 | 1:160 |
| Group 2 | | | |
| 2913 | 1:320 | 1:160 | 1:640 |
| 2914 | 1:640 | 1:320 | 1:1280 |
| 2915 | 1:640 | 1:320 | 1:1280 |
| 2916 | 1:160 | 1:160 | 1:1280 |
| 2917 | 1:160 | 1:160 | 1:160 |
| 2918 | 1:80 | 1:80 | 1:80 |
| 2919 | 1:160 | 1:160 | 1:1280 |
| 2920 | 1:160 | 1:160 | 1:640 |
| 2921 | 1:160 | 1:160 | 1:640 |
| 2922 | 1:160 | 1:160 | 1:640 |
| 2923 | 1:320 | 1:320 | 1:1280 |
| 2924 | 1:320 | NC | NC^{a} |
| Group 3 (control) | | | |
| 2925-2936 | 0^{b} | 0 | 0 |

^{a}NC = not completed (pending)
^{b}0 = <1:20

TABLE 8
Hemagglutination inhibition titers on sera of hamsters following inoculation with
West Nile virus (WNV) strain NY385-99

| GROUP | HAMSTER | Day after WNV inoculation | | | | | | |
|---|---|---|---|---|---|---|---|---|
| NUMBER | number | Day 0^{a} | Day 1 | Day 2 | Day 3 | Day 4 | Day 5 | Day 6 |
| 1 | 2901 | 1:320 | 1:640 | 1:640 | 1:640 | 1:320 | 1:320 | 1:320 |
| (E + adjuvant) | 2902 | 1:160 | 1:320 | 1:320 | 1:320 | 1:160 | 1:160 | 1:160 |
| | 2903 | 1:160 | 1:160 | 1:320 | 1:320 | 1:160 | 1:160 | 1:320 |
| | 2904 | 1:160 | 1:160 | 1:160 | 1:160 | 1:160 | 1:160 | 1:160 |
| | 2905 | 1:320 | 1:640 | 1:640 | 1:640 | 1:320 | 1:640 | 1:640 |
| | 2906 | 1:640 | 1:640 | 1:640 | 1:640 | 1:640 | 1:640 | 1:640 |
| | 2907 | 1:640 | 1:640 | 1:640 | 1:640 | 1:320 | 1:640 | 1:640 |
| | 2908 | 1:320 | 1:320 | 1:640 | 1:320 | 1:320 | 1:320 | 1:320 |
| | 2909 | 1:320 | 1:320 | 1:640 | 1:640 | 1:320 | 1:320 | 1:320 |
| | 2910 | 1:640 | 1:320 | 1:640 | 1:640 | 1:320 | 1:320 | 1:640 |
| | 2911 | 1:640 | 1:640 | 1:640 | 1:640 | 1:320 | 1:640 | 1:640 |
| | 2912 | 1:640 | 1:640 | 1:640 | 1:640 | 1:320 | 1:320 | 1:640 |
| 2 | 2913 | 1:320 | 1:320 | 1:320 | 1:160 | 1:320 | 1:320 | 1:320 |
| (E + NS1 + adjuvant) | 2914 | 1:640 | 1:640 | 1:640 | 1:640 | 1:640 | 1:320 | 1:640 |
| | 2915 | 1:640 | 1:320 | 1:640 | 1:640 | 1:320 | 1:320 | 1:320 |
| | 2916 | 1:160 | 1:160 | 1:160 | 1:320 | 1:160 | 1:160 | 1:160 |
| | 2917 | 1:160 | 1:160 | 1:160 | 1:160 | 1:160 | 1:160 | 1:80 |
| | 2918 | 1:80 | 1:80 | 1:80 | 1:80 | 1:80 | 1:40 | 1:40 |
| | 2919 | 1:160 | 1:160 | 1:160 | 1:320 | 1:160 | 1:320 | 1:320 |
| | 2920 | 1:160 | 1:160 | 1:160 | 1:160 | 1:160 | 1:160 | 1:320 |
| | 2921 | 1:160 | 1:160 | 1:160 | 1:320 | 1:160 | 1:320 | 1:320 |
| | 2922 | 1:160 | 1:160 | 1:320 | 1:320 | 1:320 | 1:320 | 1:320 |
| | 2923 | 1:320 | 1:320 | 1:320 | 1:320 | 1:320 | 1:320 | 1:640 |
| | 2924 | 1:320 | 1:160 | 1:160 | 1:160 | 1:160 | 1:160 | 1:160 |
| 3 | 2925 | 0 | 0 | 0 | 0 | 0 | 0 | 1:320 |
| (adjuvant only) | 2926 | 0 | 0 | 0 | 0 | 0 | 1:20 | 1:320 |
| | 2927 | 0 | 0 | 0 | 0 | 0 | 0 | 1:20 |
| | 2928 | 0 | 0 | 0 | 0 | 0 | 1:20 | 1:320 |
| | 2929 | 0 | 0 | 0 | 0 | 0 | 1:20 | 1:320 |
| | 2930 | 0 | 0 | 0 | 0 | 0 | 1:20 | 1:160 |
| | 2931 | 0 | 0 | 0 | 0 | 0 | 1:20 | 1:160 |
| | 2932 | 0 | 0 | 0 | 0 | 0 | 0 | 1:160 |
| | 2933 | 0 | 0 | 0 | 0 | 0 | 1:20 | 1:160 |
| | 2934 | 0 | 0 | 0 | 0 | 0 | 0 | 1:80 |
| | 2935 | 0 | 0 | 0 | 0 | 0 | 1:20 | 1:160 |
| | 2936 | 0 | 0 | 0 | 0 | 0 | 1:20 | 1:160 |

^{a}Just prior to inoculation with WNV (day 0)

TABLE 9
Level and duration of viremia in control hamsters following
intraperitoneal inoculation with 10⁴ TCID₅₀ of West Nile virus
strain NY 385-99
Group 3 (control)

| Hamster # | Day 1 | Day 2 | Day 3 | Day 4 | Day 5 | Day 6 |
|---|---|---|---|---|---|---|
| 2925 | 2.5* | 4.6 | 4.7 | 3.9 | 1.4 | 0 |
| 2926 | 2.6 | 4.5 | 4.7 | 3.8 | 1.7 | 0.7 |
| 2927 | 0 | 1.8 | 3.8 | 4.7 | 3.8 | 1.0 |
| 2928 | 1.3 | 4.6 | 4.8 | 3.6 | 1.4 | 0 |
| 2929 | 2.1 | 4.6 | 4.6 | 3.6 | 1.2 | 0.9 |
| 2930 | 2.0 | 4.5 | 4.7 | 3.6 | 1.2 | 0 |
| 2931 | 2.0 | 4.0 | 4.5 | 3.5 | 2.3 | 0.7 |
| 2932 | 0.7 | 3.6 | 4.3 | 4.1 | 3.6 | 0.7 |
| 2933 | 0.7 | 4.0 | 4.8 | 4.0 | 2.0 | 0 |
| 2934 | 0 | 3.8 | 4.6 | 4.6 | 3.6 | 1.8 |
| 2935 | 1.7 | 4.0 | 4.5 | 4.3 | 2.8 | 0.7 |
| 2936 | 2.3 | 4.2 | 4.6 | 4.8 | 2.0 | 0.7 |

*Virus titer (plaque-forming units/ml blood) expressed as log₁₀ 0 = <10^{0.7} PFU/ml

### Example 11

### Immunogenicity of Tetravalent Dengue Vaccine in Non-Human Primates

In this study, the generation of viral neutralizing and ELISA antibody titers toward the four dengue virus serotypes was tested using four antigen formulations. All four serotypes of 80% E plus NS1 were added to either a) a saponin, QS21 plus a primate targeted CpG, ODN 10103 or to b) a saponin alone (ISCOMATRIX) in concentrations as listed on Table 10.

TABLE 10

| | | Vaccine | |
|---|---|---|---|
| | | Dosing | Number of |
| Group | Antigen formulation | Days | Animals |
| 1 | 3 μg each serotype 80E + 0.3 μg NS1 | 0, 28, 67 | 2 |
| | 10 μg QS-21 + 10 μg ODN 10103 | | |
| 2 | 3 μg each serotype 80E + 0.3 μg NS1 | 0, 28, 67 | 2 |
| | 50 μg QS-21 + 50 μg ODN 10103 | | |
| 3 | 1 μg each serotype 80E + 0.1 μg NS1 | 0, 28, 67 | 2 |
| | 60 μg of ISCOMATRIX ® | | |
| 4 | 5 μg each serotype 80E + 0.5 μg NS1 | 0, 28, 67 | 2 |
| | 60 μg of ISCOMATRIX ® | | |

Serum samples were obtained prior to each vaccination and assayed for viral neutralizing antibodies as follows. Samples of diluted sera were incubated with approximately 50 plaque-forming units (pfu) of dengue virus and inoculated onto Vero cell monolayers in six-well plates (duplicate or triplicate wells). After 5 to 6 days of incubation at 35° C., the monolayers were stained with neutral red to visualize the virus-induced plaques. The dilution of serum that yielded a 50% reduction in the number of input virus plaques, determined by probit analysis, was recorded as the PRNT50 titer. Dengue virus specific IgM and IgG antibody responses were measured using a standard ELISA. Microtiter (96-well) plates were coated with each of the dengue antigens and serum dilutions were added to each well of the plate. After incubation, anti-monkey IgG conjugated with peroxidase was added, and after incubation, the plate was developed with a specific peroxidase chromogenic substrate.

Results after two or three immunizations showed that both saponin adjuvant compositions are able to generate viral neutralizing titers (“plaque reduction neutralization test”; PRNT) as well as ELISA antibody titers as shown in Tables 11, 12, and 13.

TABLE 11
Monkey Pre-Clinical Trials: Neutralizing Titers to the Dengue Virus

| Animal ID | Vaccine Formulations | Day 0 | Day 67 | Day 102 |
|---|---|---|---|---|
| | | DEN-1 | | |
| V3J | 3 μg each serotype 80E + 0.3 μg | <10 | <10 | ˜10 |
| HJC | dengue-2 NS1, 10 μg QS-21 + 10 ug ODN | <10 | <10 | <10 |
| | 10103 | | | |
| V2G | 3 μg each serotype 80E + 0.3 μg | <10 | 278 +/− 22 | ˜320 |
| AC70 | dengue-2 NS1, 50 ug QS-21 + 50 μg ODN | <10 | 53 +/− 4 | ˜200 |
| | 10103 | | | |
| AA37 | 1 μg each serotype 80E + 0.1 μg | <10 | 68 +/− 5 | Processing |
| FTH | dengue-2 NS1, 60 μg of Iscomatrix | <10 | 232 +/− 21 | Processing |
| T206 | 5 μg each serotype 80E + 0.5 μg | <10 | 34 +/− 2 | Processing |
| AJ14 | dengue-2 NS1, 60 μg of Iscomatrix | <10 | 82 +/− 4 | Processing |
| | | DEN-2 | | |
| V3J | 3 μg each serotype 80E + 0.3 μg | <10 | <10 | <100 |
| HJC | dengue-2 NS1, 10 μg QS-21 + 10 μg ODN | <10 | <10 | <100 |
| | 10103 | | | |
| V2G | 3 μg each serotype 80E + 0.3 μg | <10 | 180 +/− 12 | ˜320 |
| AC70 | dengue-2 NS1, 50 μg QS-21 + 50 ug ODN | <10 | 86 +/− 7 | ˜630 |
| | 10103 | | | |
| AA37 | 1 μg each serotype 80E + 0.1 μg | <10 | 101 +/− 11 | ˜160 |
| FTH | dengue-2 NS1, 60 μg of Iscomatrix | <10 | <80 | ˜1000 |
| T206 | 5 μg each serotype 80E + 0.5 μg | <10 | <50 | ˜320 |
| AJ14 | dengue-2 NS1, 60 μg of Iscomatrix | <10 | ˜630 | >1000 |
| | | DEN-3 | | |
| V3J | 3 μg each serotype 80E + 0.3 μg | <10 | <10 | <10 |
| HJC | dengue-2 NS1, 10 μg QS-21 + 10 μg | <10 | <10 | <10 |
| | ODN 10103 | | | |
| V2G | 3 μg each serotype 80E + 0.3 μg | <10 | ˜30 | ˜100 |
| AC70 | dengue-2 NS1, 50 μg QS-21 + 50 μg | <10 | ˜15 | ˜80 |
| | ODN 10103 | | | |
| AA37 | 1 μg each serotype 80E + 0.1 μg | <10 | ˜25 | ˜60 |
| FTH | dengue-2 NS1, 60 μg of Iscomatrix | <10 | ˜25 | ˜100 |
| T206 | 5 μg each serotype 80E + 0.5 μg | <10 | ˜10 | <50 |
| AJ14 | dengue-2 NS1, 60 μg of Iscomatrix | <10 | ˜50 | ˜80 |
| | | DEN-4 | | |
| V3J | 3 μg each serotype 80E + 0.3 μg | <10 | <10 | ˜10 |
| HJC | dengue-2 NS1, 10 μg QS-21 + 10 μg | <10 | <10 | <10 |
| | ODN 10103 | | | |
| V2G | 3 μg each serotype 80E + 0.3 μg | <10 | ˜30 | ˜100 |
| AC70 | dengue-2 NS1, 50 ug QS-21 + 50 ug | <10 | ˜50 | ˜200 |
| | ODN 10103 | | | |
| AA37 | 1 ug each serotype 80E + 0.1 μg | <10 | ˜40 | Processing |
| FTH | dengue-2 NS1, 60 ug of Iscomatrix | <10 | ˜40 | Processing |
| T206 | 5 μg each serotype 80E + 0.5 μg | <10 | <10 | Processing |
| AJ14 | dengue-2 NS1, 60 ug of Iscomatrix | <10 | ˜60 | Processing |

Some data points are still being processed.

TABLE 12
Antibody Titers in Rhesus Monkeys Immunized Twice with
Tetravalent Dengue Vaccine^{a}

| Monkey | Antibody Titer^{b} to: | | | | |
|---|---|---|---|---|---|
| ID | DEN1-80E | DEN2-80E | DEN3-80E | DEN4-80E | NS1 |
| V3J | 140 | 190 | 180 | 170 | <100 |
| HJC | 150 | 370 | 320 | 370 | <100 |
| V2G | 4200 | 5800 | 5600 | 5300 | 1100 |
| AC70 | 5000 | 5800 | 6800 | 7700 | 700 |
| AA37 | 6600 | 5800 | 7900 | 8800 | 700 |
| FTH | 6600 | 7700 | 7900 | 8400 | 3500 |
| T206 | 1300 | 1800 | 1800 | 1800 | 880 |
| AJ14 | 8400 | 9500 | 10,500 | 10,000 | 2300 |

^{a}Titers after 2 vaccinations (i.m.), given 28 days apart. Serum drawn 39 days post booster vaccination.
^{b}Titer = serum dilution yielding 50% maximum absorbance (A₄₅₀) value in ELISA assay.

TABLE 13
Antibody Titers in Rhesus Monkeys Immunized Three Times
with Tetravalent Dengue Vaccine^{a}

| Monkey | Antibody Titer^{b} to: | | | | |
|---|---|---|---|---|---|
| ID | DEN1-80E | DEN2-80E | DEN3-80E | DEN4-80E | NS1 |
| V3J | 320 | 540 | 460 | 660 | <100 |
| HJC | 180 | 430 | 410 | 370 | 100 |
| V2G | 4100 | 5100 | 5700 | 5400 | 1800 |
| AC70 | 6000 | 6800 | 9000 | 8100 | 1900 |
| AA37 | 7700 | 5000 | 9300 | 7500 | 1600 |
| FTH | 14,300 | 12,600 | 17,500 | 13,600 | 5600 |
| T206 | 2800 | 3100 | 3400 | 3200 | 1300 |
| AJ14 | 7500 | 7400 | 9500 | 8100 | 2800 |

^{a}Titers after 3 vaccinations (i.m.), given at 28 and 39 day intervals. Serum drawn 35 days post third vaccination.
^{b}Titer = serum dilution yielding 50% maximum absorbance (A₄₅₀) value in ELISA assay.

All references cited throughout the specification are expressly incorporated herein by reference. While the present invention has been described with reference to the specific embodiments thereof, it should be understood by those skilled in the art that various changes may be made and equivalents may be substituted to adapt the present invention to a particular situation. All such changes and modification are within the scope of the present invention.

What is claimed is:

1. An immunogenic composition comprising:
an effective amount of at least one recombinant flavivirus envelope protein subunit, wherein the envelope protein subunit is a portion of the envelope protein (E) that represents the portion of the envelope protein that constitutes 80% of its length starting from amino acid residue 1 at its N-terminus and which portion is a recombinantly produced protein from Drosophila cells recombinantly produced from Drosophila cells; and
an effective amount of an immunomodulating agent comprising saponin or saponin-like substance, an oligodeoxyribonucleotide, or a combination thereof, wherein the immunogenic composition induces the production of neutralizing antibodies and a cell-mediated immune response from a host provided with the immunogenic composition.

2. The immunogenic composition of claim 1, wherein the strain of the species of Flavivirus is selected from the group consisting of a strain of Dengue virus, a strain of Japanese encephalitis virus (JEV), a strain of Yellow Fever virus (YF), a strain of Tick-Borne Encephalitis virus (TBE), a strain of Saint Louis encephalitis virus (SLE), and a strain of West Nile virus (WN).

3. The immunogenic composition of claim 2, wherein the at least one envelope protein subunits comprises four envelope protein subunits derived from dengue virus serotypes 1, 2, 3, and 4.

4. The immunogenic composition of claim 1, wherein at least one recombinant flavivirus envelope protein subunits is a portion of the envelope protein (E) that represents the portion of the envelope protein that constitutes 80% of its length starting from amino acid residue 1 at its N-terminus to residue 395.

5. The immunogenic composition of claim 1, wherein the envelope protein subunit comprises six disulfide bridges at Cys1-Cys2, Cys3-Cys8, Cys4-Cys6, Cys5-Cys7, Cys9-Cys10 and Cys11-Cys12.

6. The immunogenic composition of claim 2, wherein at least one envelope protein subunit from dengue is a dimer.

7. The immunogenic composition of claim 6, wherein the dimer molecule is dimeric 80% E selected from the group consisting of: linked 80% E dimer; 80% E ZipperI; 80% E ZipperII; and 80% E Bundle.

8. The immunogenic composition of claim 7, wherein the dimeric 80% E is 80% E ZipperII.

9. The immunogenic composition of claim 8, wherein at least one dimeric envelope protein subunit is a dengue serotype 4 dimer.

10. The immunogenic composition of claim 7, wherein the leucine zipper peptide sequence further comprises a glycine-glycine-cysteine-glycine-glycine peptide at its carboxyl terminus.

11. The immunogenic composition of claim 1, further comprising at least one recombinant flavivirus non-structural protein.

12. The immunogenic composition of claim 11, wherein said recombinant Flavivirus non-structural protein is non-structural protein 1 (NS1).

13. The immunogenic composition of claim 12, wherein the NS1 is from dengue serotype 2.

14. The immunogenic composition of claim 13, wherein the NS1 is recombinantly produced and expressed in Drosophila melanogaster Schneider 2 (S2) cell lines, and is a secreted protein.

15. The immunogenic composition of claim 1, wherein said saponin is a purified derivative from Quillaja saponaria Molina bark.

16. The immunogenic composition of claim 15, wherein the purified derivative is selected from the group consisting of QS-7, QS-17, QS-18, and QS-21.

17. The immunogenic composition of claim 15, wherein said saponin is a water-soluble quillaic acid-based triterpene with an acylated 3,28-O-bisglycoside structure.

18. The immunogenic composition of claim 1, wherein said oligodeoxyribonucleotide comprises a sequence of nucleotides containing a CpG motif.

19. The immunogenic composition of claim 18, wherein said CpG motif is represented by the formula:
5′X₁X₂CGX₃X₄3′
wherein C and G are unmethylated, X₁, X₂, X₃ and X₄ are nucleotides and a GCG trinucleotide sequence is not present at or near the 5′ and 3′ termini.

20. The immunogenic composition of claim 18, wherein said CpG oligodeoxyribonucleotide is selected from the group consisting of

| | |
|---|---|
| TCCATGACGTTCCTGACGTT | (CpG ODN 1826; SEQ ID NO: 1) |
| and | |
| ATAATCGACGTTCAAGCAAG. | (CpG ODN 1760; SEQ ID NO: 2) |

21. The immunogenic composition of claim 1, wherein said oligodeoxyribonucleotide is a non-CpG oligodeoxyribonucleotide.

22. The immunogenic composition of claim 21, wherein the non-CpG oligodeoxyribonucleotide is represented by the formula:
PyNTTTTGT
wherein Py is C or T, and N is A, T, C or G.

23. The immunogenic composition of claim 21, wherein the non-CpG oligodeoxyribonucleotide is selected from the group consisting of

| | |
|---|---|
| ATAATAGAGCTTCAAGCAAG | (non-CpG ODN 1908; |
| | SEQ ID NO: 3) |
| and | |
| TCCAATGAGCTTCCTGAGTCT. | (non-CpG ODN 1745; |
| | SEQ ID NO: 4) |

24. The immunogenic composition of claim 1, wherein said oligodeoxyribonucleotide is GACGTT (hexamer CpG; SEQ ID NO: 5).

25. The immunogenic composition of claim 1, further comprising a pharmaceutically acceptable excipient.

26. A method for raising an immunogenic response from a host, comprising administering in a therapeutically acceptable manner a therapeutically effective amount of the immunogenic composition of claim 1 to said subject.
